(19) United States
(12) Patent Application Publication
(10) Pub. No.: US 2018/0369143 A1
(43) Pub. Date: Dec. 27, 2018

(54) METAL COMPLEXED THERAPEUTIC AGENTS AND LIPID-BASED NANOPARTICULATE FORMULATIONS THEREOF

(71) Applicant: BRITISH COLUMBIA CANCER AGENCY BRANCH, Vancouver (CA)

(72) Inventors: Marcel BALLY, Vancouver (CA); Ada LEUNG, Richmond (CA); Kathleen PROSSER, Quispamsis (CA); Charles WALSBY, Burnaby (CA); Mohamed WEHBE, Vancouver (CA); Malathi ANANTHA, Richmond (CA)

(21) Appl. No.: 16/061,248

(22) PCT Filed: Dec. 15, 2016
(86) PCT No.: PCT/CA2016/051480

§ 371 Date: Jun. 11, 2018

Related U.S. Application Data
(60) Provisional application No. 62/267,426, filed on Dec. 15, 2015.

(51) Int. Cl. A61K 9/127; A61K 31/551; A61K 33/24; A61K 31/4745

(52) U.S. Cl. CPC A61K 9/127; A61K 31/551; A61K 47/02; A61K 31/4745; A61K 33/24

(57) ABSTRACT

A pharmaceutical formulation for delivery of a therapeutic agent having a metal complexation moiety and a solubility in water or a metal ion solution of less than 1 mg/ml. The formulation includes the therapeutic agent and a metal ion complexed inside a lipid-based nanoparticle formulation.

### TECHNICAL FIELD

Provided herein is a formulation for delivery of one or more therapeutic agents that are poorly soluble in water or a metal-containing solution. Also provided is a pharmaceutical composition that comprises the poorly soluble therapeutic agent, CX5461.

### BACKGROUND

The aqueous solubility of organic therapeutic agents is important to their successful administration and overall efficacy. For example, the RNA polymerase inhibitor, CX5461, is presently in Phase I clinical trials as a cancer therapeutic, but has poor solubility at neutral pH. In order to overcome the low solubility at physiological pH, the drug can be provided in the form of a slurry for oral dosing or dissolved in a solution having a pH of less than 4.5 for intravenous use. With regards to the latter, these pH conditions are near the lowest that are tolerable for intravenous injection and could present potential inconsistencies in dosage due to the risk of precipitation upon introduction to physiological pH. Another example is the drug quercetin that has potential anti-cancer effects through promotion of apoptosis. Unfortunately, quercetin has been shown to exhibit limited clinical effectiveness, in part due to low oral bioavailability related to its limited solubility in aqueous solutions.

The poor solubility (herein defined as <1 mg/mL) of therapeutic agents in water is also a problem that can hinder the ability of promising new drug candidates to transition from the bench to clinical trials. In order for the efficacy of a newly discovered drug to be tested in the laboratory, such as in animal models, it often needs to be capable of administration in a water soluble form. There is a wide selection of drug candidates, such as copper complexed agents, which have been created to treat many different disease indications, including cancer, but that suffer from such poor water solubility. Without a methodology to improve the solubility properties of these promising new drug candidates, their potential to provide improvements in patient treatment may never be realized.

It is possible to use solubilizing agents to improve the solubility properties of poorly soluble therapeutic agents. There are studies that show efficacy in tumour models using solubilising agents that have been formulated at very low pH or formulated in Cremphor/DMSO/Ethanol mixtures. However, these formulations are not ideal for human use. In particular, organic solvents such as DMSO have been found to be toxic and cannot be administered to humans at concentrations above 0.5% (http://www.fda.gov/downloads/drugs/guidancecomplianceregulatoryinformation/guidances/u cm073395.pdf).

Accordingly, there is a need in the art to provide drug delivery systems for poorly soluble therapeutic agents that are suitable for parental administration. Such drug delivery systems may also allow promising therapeutics agents that are currently not in a form suitable for in vivo testing to transition from the laboratory to the clinic.

The following disclosure seeks to address one or more of the above identified problems and/or to provide useful alternatives to what is known in the art.

### SUMMARY

The inventors have discovered that a therapeutic agent that is poorly soluble (<1 mg/mL) as described herein can be efficiently incorporated into a lipid-based nanoparticulate formulation via the formation of a metal ion-drug complex. The formation of the drug-metal complex in the lipid-based nanoparticulate formulation is facilitated by chemical moieties on the therapeutic agent, which may include the following groups: S-donor, O-donor, N, O donor, Schiff bases, hydrazones, P-donor phosphine, N-donor or combinations thereof.

The method described herein for producing the lipid-based nanoparticulate formulation can potentially serve as a platform approach suitable for a wide range of sparingly soluble agents of therapeutic interest. Furthermore, with the existence of other donor systems known in the art, the method could be applied to a broad range of drugs and drug candidates with a variety of structures, sizes and metal-binding moieties.

Moreover, according to certain embodiments, the lipid-based nanoparticulate formulations prepared as described herein have been found to be stable over time. For example, the nanoparticulate formulations described in certain embodiments may be stable with respect to particle size, surface charge and complex-to-lipid ratio for at least 30 days at 4° C. In addition, the method for preparing the lipid-based nanoparticulate formulation herein is scalable and suitable for manufacturing a pharmaceutical product. As described herein, the lipid-based nanoparticulate formulation may be a lipid vesicle, also referred to herein as a liposome.

Thus, according to one embodiment, there is provided a pharmaceutical formulation for delivery of a poorly soluble therapeutic agent, the formulation comprising: a metal ion and the poorly soluble therapeutic agent inside a lipid-based nanoparticulate formulation, which sparingly soluble therapeutic agent has a solubility of less than 1 mg/mL when in either water or in a solution of the metal ion, the therapeutic agent comprising a metal complexation moiety, and wherein the complexation moiety complexes with the metal ion inside the lipid-based nanoparticulate formulation. The poorly soluble therapeutic agent may have a pKₐ of at least 8. According to any one of the foregoing embodiments, the lipid-based nanoparticulate is a liposome. In another embodiment, the poorly soluble therapeutic agent is non-pH gradient loadable into the liposome.

According to a further embodiment of the invention, there is provided a method for producing a pharmaceutical formulation for delivery of a poorly soluble therapeutic agent, the method comprising: (i) providing a pre-formed liposome comprising a phospholipid bilayer and a metal ion that complexes with the poorly soluble therapeutic agent; (ii) providing a poorly soluble therapeutic agent in the solution external to the liposome, the therapeutic agent comprising a metal ion complexation moiety; and (iii) allowing the therapeutic agent to move across the phospholipid bilayer of the liposome into the liposome, wherein the poorly soluble therapeutic agent has a solubility of less than 1 mg/mL in either water or a solution containing the metal ion.

According to any one of the foregoing embodiments, the metal may be a transition metal or a Group IIIb metal. The drug-to-lipid ratio may be at least 0.2:1, or at least 0.3:1.

In another embodiment there is provided a liposome formulation comprising a liposome, wherein the liposome comprises a therapeutic agent selected from clioquinol, diethyldithiocarbamate, quercetin, and CX5461 and wherein the liposome comprises a metal ion that complexes with the therapeutic agent.

According to any one of the foregoing embodiments, the poorly soluble therapeutic agent is not mitoxantrone, doxorubicin, epirubicin, daunorubicin, irinotecan, topotecan, vincristine, vinorelbine or vinblastine.

Additional embodiments disclosed herein are based on the discovery that the poorly soluble therapeutic agent, CX5461, having Formula I shown below displays enhanced water solubility at a physiological pH range when complexed with a metal ion. The enhanced solubility of copper complexed CX5461 confers desirable pharmacokinetic properties such as improved absorption, bioavailability and/or the ability to deliver higher dosages of the therapeutic agent.

Thus, according to certain embodiments of the invention, there is provided a pharmaceutical composition comprising CX5461 having the following Formula I:

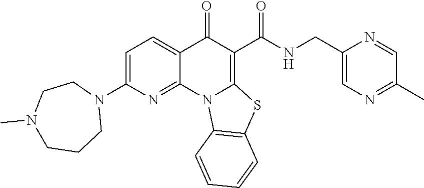

Formula I

wherein the CX5461 is complexed with a metal ion.

The foregoing pharmaceutical composition may have a pH in the range of between 5 and 9, or any range therebetween.

The pharmaceutical composition may comprise CX5461, the metal ion and a carrier for the therapeutic agent such as a pharmaceutically acceptable excipient or diluent. In one embodiment, the pharmaceutical composition comprises a lipid-based nanoparticulate formulation such as a liposome having encapsulated therein the CX5461 complexed with the metal ion. However, it should be appreciated that the pharmaceutical composition may contain CX5461 in free form. That is, the CX5461 need not be incorporated in liposomes or other similar delivery vehicle.

Further aspects of the invention will become apparent from consideration of the ensuing description of preferred embodiments of the invention. A person skilled in the art will realise that other embodiments of the invention are possible and that the details of the invention can be modified in a number of respects, all without departing from the inventive concept. Thus, the following drawings, descriptions and examples are to be regarded as illustrative in nature and not restrictive.

### BRIEF DESCRIPTION OF FIGURES

FIG. 1 illustrates the role of diethyldithiocarbmamate (DDC) in cancer therapy through administration with copper. (A) Disulfiram metabolism to DDC and complexation of DDC with copper (Cu) (II). (B) Cytotoxicity curves for DSF (●) and DSF+CuSO₄ (▪). (C) Cytotoxicity curves for DDC (●) and DDC+CuSO₄ (▪) obtained with IN CELL Analyzer in U87 glioblastoma cell lines. (D) IC₅₀ values for U251, MDA-231-BR, A549 cancer cell lines and HBEpC (normal bronchial epithelial cells) for DDC and Cu(DDC)₂ (n.d.=no data). (E) Pictorial representation of DDC, CuSO₄ and Cu(DDC)₂ solutions in water. Data points are given as mean ±SEM.

FIG. 2 is a graphical depiction of the copper-complex based loading method. The loading scheme can be seen graphically at the top of the figure in which Cu²⁺ lipid-based nanoparticulate (LNP) formulations are mixed with the therapeutic agent, diethyldithiocarbmamate (DDC). The resulting LNPs are produced with the Cu-complex suspended inside. This can be seen in the UV spectra at the bottom of the figure that shows a shift in absorbence at 435 nm.

FIG. 3 shows the loading of DDC into 300 mM Cu²⁺-DSPC/Chol (55:45) liposomes. (A) Pictorial representation of DDC (5 mg/mL) loading into 20 mM CuSO₄-Liposomes for 1 hour at 25° C. (B) Cu(DDC)₂ drug loading time course for 1 hour at 4(●), 25(▪) and 40(▴)° C. for DSPC/Chol LNPs (20 mM) and DDC (5 mg/mL). (C) Cu(DDC)₂ drug loading time course for a pH gradient and pH gradient free system both in SH buffer at pHs 7.4 and 3.5 respectively. (D) Cu(DDC)₂ ratio as a function of changing the theoretical D/L (drug-to-lipid ratio) that can be obtained. All measurements were performed using a fixed lipid concentration of 20 mM and altering DDC content. (E) Cryo-Electron Microscopy of empty DSPC/Chol (55:45) LNPs (top) and Cu(DDC)₂ loaded LNPs (bottom). (F) Size of both CuSO₄-LNPs and Cu(DDC)₂-LNPs by quasi-electric light scattering and cryo-electron microscopy. Data points are given as mean ±SEM.

FIG. 4 shows data characterizing copper-complex drug loading into liposomes. (A) Copper-to-lipid (black) and Cu(DDC)₂ to lipid ratios (grey) of 300 mM Cu²⁺-DSPC/Chol/(DSPE-PEG₂₀₀₀) liposomes at different concentrations of DSPE-PEG₂₀₀₀. (B) Cu(DDC)₂ in liposomes as a function of the amount of copper used for rehydration. Copper-to-lipid (black) and Cu(DDC)₂-to-lipid ratios (grey) are shown. (C) Linear regression analysis on the amount of copper trapped vs the Cu(DDC)₂ complex formed .(R²=0.9754). Data points are given as mean ±SEM.

FIG. 5 shows donor systems that can be used in copper(II)-complex loading. The copper is able to form complexes with drugs containing S, O, N and mixed donor systems. Diethyldithiocarbamate (DDC), Quercetin (Qu), Clioquinol (CQ) and CX5461 are shown in the figure as examples of drugs that can be loaded into liposomes. Each was loaded into DSPC/Chol liposomes containing 300 mM CuSO₄ at 25, 50, 40 and 50° C. respectively.

FIG. 6 shows the diagnostic metal absorption bands for CX5461 and copper, CX5461 and zinc and CX5461 alone that were monitored in the UV-Vis spectra. The graph shows the absorbance verses wavelength (nm) for copper only and copper in combination with CX5461 at Cu to drug ratios of 1:0.4, 1:0.8, 1:1.2 and 1:1.6.

FIG. 7 shows the proton NMR spectra of CX5461 and copper (top), CX5461 and zinc (middle) and CX5461 alone (bottom).

FIG. 8 shows the coordination complex formed by CX5461 with copper (II) and zinc (II) ions. The region labelled A in the ¹H NMR spectrum shows signal broadening due to paramagnetism of a Cu (II) ion. A zinc NMR sample (10 mM Zn(II)Cl₂ with 5 mM of CX5461 in D₂O at pD 6) exhibits a significant difference in carbon chemical shifts when compared to CX5461 in phosphate buffer in the absence of a metal cation. This NMR analysis demonstrated significant shifts in carbons x and z (downfield shifts) and carbons y and aa (upfield shifts). This suggests coordination of the M²⁺ cation through the ortho-N position of the pyrazine ring. Further NMR experiments were carried out to determine the effects of coordination to the paramagnetic Cu²⁺ on the relaxation rate. These results demonstrated again not only the strong association of metal cations to the pyrazine ring, but also the significant effect on the aromatic core. Without being bound by theory, the data suggests multi-dentate coordination to the carbonyl oxygen k, the bridging nitrogen v, and the pyrazine ortho-N. These results were further corroborated by density functional theory calculations, and indicate that the spin density of the copper(II) extends across the pyrazine and to the aromatic system when in this binding pocket.

FIG. 9 shows the Cu Electron Paramagnetic Resonance (EPR) spectra of CuSO₄ with CX5461 at a copper-to-drug ratio of 1:0.5 (top curve) and 1:1 (bottom curve).

FIG. 10 shows that CX5461 forms a complex with a metal (copper). (A) The structure of CX5461. (B) At equal concentrations, copper sulfate (CuSO₄) and CX5461 alone dissolved in NaH₂PO₄ are colourless solutions as shown in the test tubes at the left and in the middle, while the contents of the test-tube containing Cu-CX5461 (right test-tube) are a darker in colour. During the experiment, this was observed as a blue colour. (C) Results of a 72-hour cytotoxicity assay in the presence of CX5461, Cu and Cu-CX5461 in H460 cells (non-small cell lung cancer) and MV-4-11 cells (biphenotypic B-myelomonocytic leukemia). The results are shown as the fraction of affected cells (Fa) vs drug concentration (μM). (D) IC₅₀ values were compared using ANOVA followed by Dunnett's multiple comparisons test and no statistical significance was detected with each cell line with α=0.05.

FIG. 11 provides data showing that CX5461 can be encapsulated into liposome formulations using copper in the internal loading medium. (A) CX5461 dissolved in sodium phosphate at pH 3.5 as loaded into copper-containing liposome formulations at 4° C., room temperature, 40° C., 50° C. and 60° C. (B) Shows the drug loading efficiency (%) of CX5461 vs the copper concentration (mM) in the liposome. (C) The leftmost test-tube shows a CuSO₄ liposome formulation before drug loading and the rightmost test-tube, which is darker in colour, shows copper-containing liposomes loaded with CX5461.

FIG. 12 provides data showing that CX5461-containing liposomes encapsulated with copper are stable for at least 3 weeks. (A) The drug-to-lipid ratio (D/L; A), and (B) particle size, and polydispersity of the formulation were determined on days 1, 3, 5, 7, and 21, with day 1 being the day that the formulation was prepared.

FIG. 13 demonstrates the enhanced pharmacokinetics (PK) profile and in vivo activity of CX5461 when encapsulated in copper-containing liposomes. (A) shows the CX5461 concentration (μg/mL) as a function of time post-injection (h). (B) shows tumour volume (mm³) as a function of days post-inoculation.

FIG. 14 demonstrates the solubility of quercetin in aqueous buffers. (A) shows the solubility of quercetin in water and HEPES buffer saline, wherein 10 mg of quercetin powder was mixed at 60° C. (or 22° C.) for 60 minutes in 2 mL of the respective buffers. (B) shows quercetin dissolved at 60° C. for 60 minutes in HBS. The time points for dissolution were 5, 10, 15, 30 and 60 minutes.

FIG. 15 shows loading of quercetin into liposomes at different temperatures. (A) illustrates that quercetin is a three-ringed flavonoid. (B) Drug-to-lipid ratios of quercetin loading into copper-containing liposomes over 60 minutes with time points at 5, 10, 30 and 60 minutes at 22° C., 40° C., 50° C. and 60° C. (C) Quercetin-loaded liposomes (600 μL/tube) collected through mini spin columns at each respective time point. Data points represent the mean ±SEM (n=3).

FIG. 16 shows loading of quercetin at various copper concentrations and at different intra-liposomal pH values. (A) Quercetin powder was loaded into liposomes of varying internal CuSO₄ concentration at 60° C. for 60 minutes. (B) Loaded drug-to-lipid ratio is plotted against copper-to-lipid ratio of quercetin encapsulated liposomes with varying internal CuSO₄ concentrations. (C) Quercetin was encapsulated into copper-containing liposomes (100 mM copper gluconate) at an internal buffer pH of 3.5 and 7.4 and into copper-free liposomes (300 mM citric acid) at an internal pH of 3.5 and 7.4. Data points represent the mean ±SEM (n=3).

FIG. 17 shows quercetin encapsulation into CuSO₄-containing liposomes and copper gluconate-containing liposomes. (A) Quercetin was loaded into 100 mM and 300 mM CuSO₄ and 100 mM copper gluconate. (B) Copper-to-lipid ratios of loading of quercetin into 100 mM and 300 mM CuSO₄ and 100 mM copper gluconate liposomes at 60° C. Data points represent the mean ±SEM (n=3).

FIG. 18 shows loading of quercetin at various internal copper gluconate concentrations. (A) Quercetin was loaded into liposomes of varying internal copper gluconate concentrations (0, 10, 25, 75 and 100 mM copper gluconate) at 60° C. for 60 minutes. (B) Loaded drug-to-lipid ratios were plotted against copper-to-lipid ratios of quercetin encapsulated liposomes with varying internal copper gluconate concentrations (0, 10, 25, 75 and 100 mM copper gluconate) at 60° C. for 60 minutes. Data points represent the mean ±SEM (n=3).

FIG. 19 shows quercetin complexation with copper. (A) Quercetin and the complexes it forms with copper can be visualized via spectrophotometric (UV absorbance) measurements in methanol, where quercetin peaks at 372 nm and quercetin-copper complex peaks at 441 nm. (B) CuSO₄ and copper gluconate were titrated against a fixed quercetin concentration (5 μg/mL) at absorbance wavelength of 441 nm at copper-to-quercetin ratios of 1:8, 1:4, 1:2, 1:1, 2:1, 4:1 and 8:1. (C) Possible molecular structures of copper-quercetin complexes with copper gluconate (left panel) and CuSO₄ (right panel) are shown.

FIG. 20 is the in vitro release of quercetin encapsulated in CuSO₄ liposomes and quercetin encapsulated in copper gluconate liposomes in fetal bovine serum (FBS). Formulations were incubated in 80% Fetal bovine serum at 37° C. over 24 hours. Data points represent the mean ±SEM (n=3).

FIG. 21 shows the pharmacokinetics profiles of quercetin-loaded 300 mM CuSO₄ and copper gluconate liposomes in vivo. Female RAG2m mice were injected intravenously with a single bolus dose of liposomal quercetin at 50 mg/kg. (A) shows plasma concentrations of quercetin over a 24 hours period following drug administration and (B) shows lipid concentrations over the same period. Data are plotted as ±SEM (n=4). The resulting drug-to-lipid ratio (C) and copper-to-lipid ratio (D) are plotted as an indication of drug and copper release from the liposomes over time. LNP-CuSO₄-Q=CuSO₄ liposomes loaded with quercetin. LNP-CuG-Q =CuG liposomes loaded with quercetin. Data points represent mean ±SEM (n≥4).

FIG. 22 demonstrates the anticancer activity of copper clioquinol (Cu(CQ)₂) in cancer cell lines. Cytotoxicity curves for CQ (-●-) and Cu(CQ)₂ (-▪-) were obtained for (A) A2780-S, (B) A2780-CP (C) A549, (D) U251 and (E) MV-4-11 cells. Cell viability for (A-D) was obtained using the IN CELL analyzer where viability was assessed based on loss of plasma membrane integrity 72 hours following treatment; i.e., total cell count and dead cell count were determined using Hoechst 33342 and ethidium homodimer staining, respectively. MV-4-11 cell viability was measured through metabolic activity using PrestoBlue.

FIG. 23 Formation of copper clioquinol (CQ) into DSPC/Chol (55:45) liposomes prepared with encapsulated 300 mM CuSO4. (A) Photograph of solutions consisting of CQ (10 mg/mL) added to CuSO₄-containing DSPC/Chol (55:45) liposomes (20 mM liposomal lipid) over a 1 hour time course at 40° C. (B) Formation of Cu(CQ)₂ inside DSPC/Chol liposomes (20 mM) as a function of time over 1 hour at 4(●), 25(▪) 40(▴) and 50(▾)° C. following addition of CQ at a final CQ concentration of (15 mM). The Cu(CQ)₂ was measured using a UV-Vis spectrophotometer and liposomal lipid was measured through use of a radiolabeled lipid (³H-CHE). (C) Measured Cu(CQ)₂ as a function of increasing CQ added, represented as the theoretical Cu(CQ)₂ to total liposomal lipid ratio; where the lipid concentration was fixed at 20 mM and final CQ concentration was varied. (D) In vitro stability of the Cu(CQ)₂ formulation over 24 hours in 80% fetal bovine serum. All data are plotted as mean ±SEM.

FIG. 24 Cu(CQ)₂ and copper lipsome elimination profiles upon intravenous injection in CD-1 mice. Cu(CQ)₂ dose was 30 mg/kg and the associated lipid dose was 115.6 mg/kg. Copper liposomes were injected at the same lipid dose of 115.6 mg/kg. (A) Clioquinol plasma concentration over 24 hrs. (B) Clioquinol-to-lipid ratio over 24 hrs for the Cu(CQ)₂ formulation. (C) Cu(CQ)₂ (●) and copper (▪) liposomes, Cu²⁺ was measured using AAS over 24 hrs. (D) Copper to lipid ratio over 24 hrs for liposomes prepared in 300 mM copper sulfate or with associated Cu(CQ)₂. (E) The liposomal lipid concentration was measured using scintillation counting of ³H-CHE. All data are plotted as mean ±SEM.

FIG. 25 The Cu(CQ)₂ formulation was assessed for efficacy in a subcutaneous U251 tumour model. (A) Maximum tolerated dose of Cu(CQ)₂ was determined for intravenous (▪) and intraperitoneal (▴) injection in CD-1 mice. (B) Subcutaneous U251 tumour growth in Rag2M mice after treatment with Vehicle (●), Cu(CQ)₂ i.v. 30 mg/kg (▪) Q2D×2 weeks or Cu(CQ)₂ i.p. 15 mg/kg (▴) QD (M-F)×2 weeks. (C) The Kaplan-Meier survival curve is plotted and a statistically significant increase in survival was seen for Cu(CQ)₂ i.v. 30 mg/kg (-) Q2D×2 weeks or Cu(CQ)₂ i.p. 15 mg/kg ( [symbol] ) QD (M-F)×2 weeks. The symbol “*” indicates a statistically significant difference (p<0.05).

FIG. 26 Clioquinol metal complex cytotoxicity in A2780-S (ovarian cancer) cells. (A) Cytotoxicity curves for CQ (-●-), Cu(CQ)₂ (-▪-) and Zn(CQ)₂ (-♦-) were obtained using the INCELL analyzer where viability was assessed based on loss of plasma membrane integrity 72 hours following treatment; i.e., total cell count and dead cell count were determined using Hoechst 33342 and ethidium homodimer staining, respectively. Results are given as mean ±SEM (B) IC₅₀ values of CQ and metal complexes in A2780-S (IC₅₀±95% CI).

FIG. 27 shows the in vivo testing of Cu(DDC)₂, Cu(CQ)₂, CuQu and Cu-CX5461 in female CD-1 mice after single intravenous bolus injection for toxicity and pharmacokinetics. Mice were injected with a single injection of 15 mg/kg Cu(DDC)₂(●), 30 mg/kg Cu(CQ)₂ (▪), 70 mg/kg CuQu (▴) and 50 mg/kg Cu-CX5461(▾). (A) A graph showing percent change in body weight vs. time (day) measured for 14 days post injection (n=3) mice. (B) A graph showing percent injected dose vs. time of Cu-complex formulations at the selected time points (1, 4, 8 and 24 hrs) mice (n=4).

FIG. 28 shows the dose to achieve 95% cell kill (μM) in vitro for CX5461 and irinotecan (CPT11) as single agents (filled bars) and in combination (bars with no fill).

FIG. 29 shows an in vitro cytotoxicity assay evaluating the combination effect of irinotecan and quercetin. (A) the left panel shows the cytotoxic effects of quercetin (Quer) and/or irinotecan (CPT11) for A549 lung cancer cells and the right panel shows BxPC3 pancreatic cancer cells. For combination treatments, Quer and CPT11 were added at ratios of 1:2.5 (CPT11:Quer) for A549 and 1:18 (CPT11:Quer) for BxPC3. The dose response curve for the combination was plotted based on CPT11 concentrations. (B) shows the IC₅₀ values following 72 hours of drug exposure. (C) the Combination Indices (CI) derived from the dose response of the combination treatment are plotted against treatment effectiveness where a fraction affected of 1 indicates 100% cell kill. CI>1 =antagonistic, CI=1 is additive and CI<1 is synergistic. All data are plotted as mean ±SEM (n≥3).

### DETAILED DESCRIPTION

Therapeutic Agent(s)

The poorly soluble (<1 mg/mL) therapeutic agent is capable of complexing with a metal ion. In order for such complexation to occur, the therapeutic agent comprises a complexation moiety, such as a moiety selected from an S-donor, O-donor, N, O donor, a Schiff base, hydrazones, P-donor phosphine, N-donor or a combination thereof. In another embodiment, the moiety is a hard electron donor. Other moieties known to those of skill in the art suitable for complexation with a metal ion are included within the scope of the invention as well. This includes, but is not limited to, any ligands that are capable of donating electrons to the d orbitals of a metal.

As noted, the poorly soluble therapeutic agent selected for incorporation in the lipid-based nanoparticulate formulation is also considered poorly soluble in solution prior to or after complexation with the metal ion. By this it is meant that the poorly soluble therapeutic agent in free form has a solubility of less than 1 mg/mL in either water or a solution of the metal ion that complexes with the therapeutic agent. Solubility of the therapeutic agent in water or in the presence of the metal ion is measured at conditions of physiological pH and temperature after 60 minutes of incubation under these conditions. The concentration of the metal ion in the metal ion solution is between 10 mM to 500 mM. If the therapeutic agent has a solubility of less than 1 mg/mL at any concentration of metal ion within the foregoing range, under the conditions specified, then it is considered poorly soluble for purposes herein. The metal ion in the metal ion solution corresponds to the metal ion incorporated in the lipid-based nanoparticulate formulation.

In one embodiment, the solubility of the poorly soluble therapeutic agent is less than 1, 0.95, 0.90, 0.85, 0.80, 0.75, 0.70 or 0.65 mg/mL.

The therapeutic agent (also referred to herein simply as a “drug”) is capable of exerting an effect on a target, in vitro or in vivo to treat or prevent a disorder or disease. In one embodiment, the therapeutic agent is an anti-cancer therapeutic agent.

Non-limiting examples of poorly soluble therapeutic agents include 8-hydroxyquinoline, pyrithione, plumbagin, ciclopirox, fusaric acid, clioquinol, ciprofloxacin, nalidixic acid, oxflacin, lomafloxacin, oxolinic acid, norfloxacin, enoxacin, piromidic acid, metformin, moroxidin, phenformin, ethambutol, diflunisal, flumequine, minocycline, mimosine, apigeninn, mycophenolic acid, chrysin, dioxygenzone, mesalamine, isoniazid, pyrazinamide, ethionamide, diethyldithiocarbamate, quercetin, naproxen, diclofenac, indomethacin, ketoprofen, mefenamic acid, acetylsalicylic acid, piroxicam, acemetacin, valproic acid, CX3543 and CX5461.

According to one embodiment of the invention, the poorly soluble therapeutic agent is not mitoxantrone, doxorubicin, epirubicin, daunorubicin, irinotecan, topotecan, vincristine, vinorelbine or vinblastine. These are therapeutic agents that are known to be pH gradient loadable into liposomes, have a solubility of >1 mg/mL and can also bind metal ions.

In one embodiment, the therapeutic agent is a flavonol or a quinolone. In another embodiment, the therapeutic agent is selected from diethyldithiocarbamate (DDC), quercetin (Qu), clioquinol (CQ), CX3543 (quarfloxacin) and CX5461. DDC is an X-donor, Qu an O-donor, and CQ is an N, O donor. Chemical structures for DDC, Qu, CQ and CX5461 are provided in FIG. 5. In another embodiment, the therapeutic agent is CX3543. In a further embodiment, the therapeutic agent has a pKₐ that is greater than 8. In another embodiment, the therapeutic agent has a pKₐ greater than 8.2, greater than 8.4 or greater than 8.6.

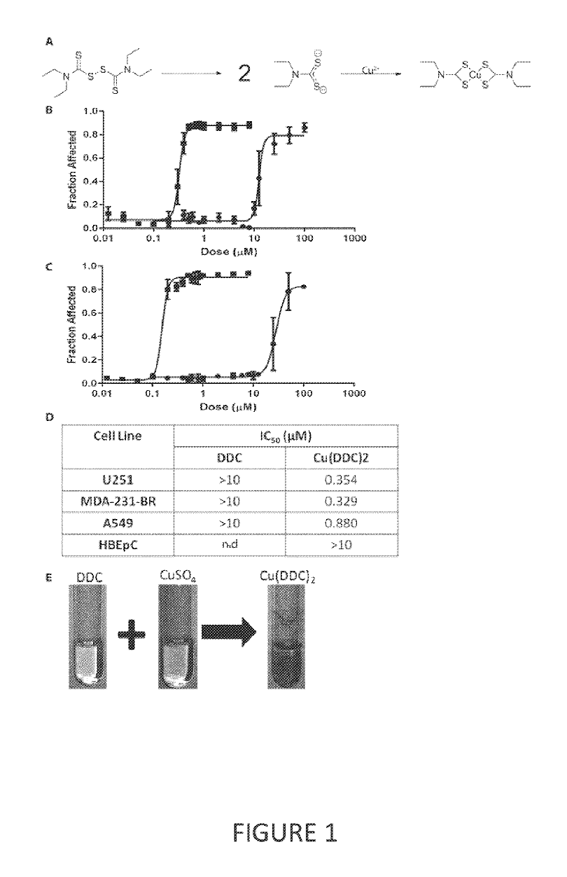

Diethyldithiocarbamate (DDC) is known to be an active metabolite generated following administration of disulfiram (DSF) used to treat chronic alcoholism. DSF inhibits acetaldehyde dehydrogenase 1 (ALDH1) and is a drug of interest for use in the treatment of human immunodeficiency virus (HIV) and cancer. DSF has been used clinically and there are studies that explore its pharmacokinetic properties. DSF is metabolized to DDC, which is a metal chelator. DDC forms a copper complex at a 2:1 mole ratio (DDC:Cu²⁺), a reaction that may be detected by the eye as a brown precipitate forms (see, for example, FIG. 1A).

Quercetin (Qu) is an antioxidant that may protect against damages associated with oxidative stress induced by free radicals or reactive oxidative species. In addition, Qu has been shown to exhibit anti-cancer capabilities in various cancer models by induction of apoptosis signaling cascades. For example, in studies with A549 lung cancer cells, human glioma cells and human hepatoma cells, quercetin was found to induce cancer cell death by downregulation of anti-apoptotic proteins such as Bcl-2, AKT and metallopeptidases 9 and upregulation of pro-apoptotic proteins such as Bax and those involved in the caspase cascade. In addition to acting as a single anti-cancer agent, quercetin may sensitize cancer cells to existing anti-cancer therapeutics.

Clioquinol (CQ) is an analogue of 8-hydroxyquinoline and is an FDA approved antibacterial agent. It forms a Cu(II) complex which inhibits proteosome function and is a copper ionophore.

CX5461 is a RNA polymerase inhibitor being evaluated in clinical trials and its use exemplifies the versatility of this method as CX5461 is a high molecular weight compound with many functional groups capable of binding copper.

As discussed below, more than one therapeutic agent may be encapsulated in the liposome. The additional therapeutic agent(s) may have a solubility of more than or less than 1 mg/mL in water or a metal ion containing solution.

Lipid-Based Nanoparticulate (LNP) Formulation

As discussed, the therapeutic agent(s) is encapsulated in a lipid-based nanoparticulate formulation (LNP). The lipid-based nanoparticulate formulation includes micro- or nano-particles that includes at least one amphipathic layer that comprises lipids and includes a liposome. A liposome is a vesicle comprising a bilayer having amphipathic lipids enclosing an internal solution. The liposome may be a large unilamellar vesicle (LUV), which can be prepared as described below using extrusion. In one embodiment, the diameter of the liposome may be between 60 nm and 120 nm or between 70 and 110 nm.

The liposome may comprise lipids including phosphoglycerides and sphingolipids, representative examples of which include phosphatidylcholine, phosphatidylethanolamine, phosphatidylserine, phosphatidylinositol, phosphatidic acid, pahnitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Other compounds lacking in phosphorus, such as sphingolipid and glycosphingolipid families are also encompassed by certain embodiments. The phospholipids may comprise two acyl chains from 6 to 24 carbon atoms selected independently of one another and with varying degrees of unsaturation. Additionally, the amphipathic lipids described above may be mixed with other lipids including triacylglycerols and sterols. As would be appreciated by those of skill in the art, lipids that interfere with liposome formation in the presence of a metal should typically be avoided. Whether or not a given lipid is suitable for liposome formation in the presence of a metal ion can be determined by those of skill in the art.

In one embodiment, the liposome comprises the lipids 1,2-distearoyl-sn-glycero-3-phosophocholine (DSPC)/Cholesterol. The precise ratios of the lipids may vary as required. A non-limiting example of a suitable ratio of DSPC/Cholesterol is 55:45 mol:mol. The liposomes may also comprise a hydrophilic polymer-lipid conjugate. The hydrophilic polymer may be a polyalkylether, such as polyethylene glycol. The hydrophilic polymer-lipid conjugate is generally prepared from a lipid that has a functional group at the polar head moiety that is chemically conjugated to the hydrophilic polymer. An example of such a lipid is phosphatidylethanolamine. The inclusion of such hydrophilic polymer-lipid conjugates in a liposome can increase its circulation longevity in the bloodstream after administration. The hydrophilic polymer is biocompatible and has a solubility in water that permits the polymer to extend away from the liposome outer surface. The polymer is generally flexible and may provide uniform surface coverage of the liposome outer surface. In addition, it has been found herein that the inclusion of such a hydrophilic polymer-lipid conjugate can increase the amount of the transition metal encapsulated in the liposome. This can be used as a methodology to increase the amount of the therapeutic agent encapsulated in the liposome.

In one embodiment, the liposome may include a hydrophilic polymer, such as polyethylene glycol (PEG) at between 1 and 20 mol % or between 2 and 10 mol %. An example of a formulation comprising PEG is DSPC/CHOL/PEG (50:45:5, mole ratio) or DSPC/PEG (95:5, mole ratio). The specific ratios of the lipids, however, may vary according to embodiments visualized by persons skilled in the art.

The liposome comprises a metal ion that is capable of forming a complex with the therapeutic agent. The metal ion may be an ion of a transition metal or a Group IIIb metal. The transition metal may be from Group 1B, 2B, 3B, 4B, 5B, 6B, 7B and 8B (groups 3-12). Examples of transition metals include copper, zinc, manganese, iron, cobalt and nickel. The Group IIIb metal is from the boron family, which includes boron, aluminum, gallium, indium, thallium and nihonium. In one embodiment, the metal is in the 2⁺ oxidation state. In another embodiment, the metal has d-orbitals. Typically, the metal ion is incorporated inside the liposome during its preparation. In another embodiment, the liposome is formed with a lipid having a chelating group that binds a metal ion, as described below. In this exemplary embodiment, the metal that is inside the liposome may be associated with a lipid that makes up an inner leaflet of the bilayer.

Liposomes can be prepared by any of a variety of suitable techniques known to those of skill in the art. An example of one suitable method involves cycles of freeze-thaw and subsequent extrusion of lipid preparations. According to one such method, lipids selected for inclusion in a liposome may be dessicated and dissolved in a solvent, such as an organic solvent, at a desired ratio. After removal of the solvent, the resultant lipids are hydrated in an aqueous solution. The solution in which the lipids are hydrated forms the internal solution of the liposomes. Subsequently the hydrated lipids may be subjected to cycles of freezing and thawing. The hydrated lipids are passed through an extrusion apparatus to obtain liposomes of a defined size. The size of the resulting liposomes may be determined using quasi-electric light scattering (e.g., using a NanoBrook ZetaPALS Potential Analyzer).

As discussed, the liposomes may be prepared so that they comprise an internal solution comprising the metal ion. For example, when preparing liposomes by freeze-thaw and subsequent extrusion as described above, the lipids are hydrated in a solution comprising a metal ion. However, the liposomes so formed will comprise the metal ion not only in the internal solution of the liposomes, but also in the external solution. Unencapsulated metal ion is removed from the external solution of the liposome prior to loading of the one or more therapeutic agents. For example, the external copper or zinc-containing solution may be exchanged with a solution containing substantially no copper or zinc ions by passage through a column equilibrated with a buffer. Other techniques may be employed such as centrifugation, dialysis, the addition of a chelating agent, such as EDTA (to chelate the metal) or related technologies. Typically the solution that exchanges with the metal-containing solution is a buffer, although other solutions may be used as desired. The liposomes may be subsequently concentrated to a desired lipid concentration by any suitable concentration method, such as by using tangential flow dialysis.

In one embodiment, the solution external to the liposome contains substantially no metal ions that complex with the poorly soluble therapeutic agent. By this it is meant that the concentration of metal ions in the external solution is less than that of the metal ion concentration in the liposome, of less than one fifth of the concentration of metal ion in the liposome. Alternatively, or in addition, the external solution may comprise a chelating agent that chelates with the metal ions.

As noted, the metal ion may be encapsulated in the liposome as a metal salt. Examples include copper sulfate, copper chloride or copper gluconate. Likewise, a zinc salt may be enclosed in the lipid bilayer. An example of a suitable zinc salt is zinc sulfate.

The metal ion and poorly soluble therapeutic agent are inside the lipid-based nanoparticulate formulation. That is, the metal ion will be complexed with the therapeutic agent inside the nanoparticulate in the internal solution of the particulate formulation. As noted, in one embodiment, this includes association of the metal ion with a lipid on an internal leaflet of a lipid bilayer. For example, the liposome could be formed using one or more lipids modified with a chelating group. The chelating group may bind with a metal and the metal in turn could complex with a complexation moiety present on the therapeutic agent.

The liposomes comprising the metal ion are incubated with the one or more therapeutic agents to facilitate uptake thereof. The therapeutic agent may be added in any suitable form, including as a powder or as a solution. If the therapeutic agent is insoluble in water, it can be added as a powder. The amount of free therapeutic agent in solution can subsequently be increased by increasing the temperature. Incubation of the pre-formed liposomes with the one or more therapeutic agents is performed under conditions sufficient to allow the poorly soluble therapeutic agent to move across the phospholipid bilayer of the liposome into the internal solution thereof. Such a method is referred to by those of skill in the art as “loading”.

Movement of the therapeutic agent across the phospholipid bilayer of the liposome during loading may occur independently of any pH gradient across the bilayer. The loading may, however, be dependent on other factors. As will be appreciated, the loading conditions can be readily selected by those of skill in the art to achieve a desired rate of loading. For example, the diffusion of the therapeutic agent across the bilayer may be dependent on the temperature and/or lipid composition of the liposome. Using Qu as a non-limiting example to illustrate, this compound may be added as a powder to the pre-formed copper liposomes. The amount of Qu in free solution, albeit low, will increase with increasing temperature. Solubilized Qu will be free to move across the liposomal lipid bilayer (from the outside to the inside), and the permeability of Qu across the membrane will be dependent on the lipid composition and temperature.

Once incorporated with the liposome, the poorly soluble therapeutic agent will form a complex with the metal ion. Without being bound by theory, the formation of the drug-metal complex may be characterized as an inorganic synthesis reaction. In certain embodiments, the uptake of drug during the loading reaction is visualized as a colour change as many metal complexed therapeutic agents have different spectral characteristics that can be detected by eye. For example, a colour change to purple, brown, green or yellow can be observed during loading with copper. By formulating complexes through such an inorganic synthesis reaction occurring within the internal solution of the liposome, a high drug-to-lipid ratio may be attained. For example, the drug-to-lipid ratio may be about 0.1:1 to about 0.6:1 (mol:mol), 0.15:1 to 0.5:1 (mol:mol) or 0.2:1 to 0.4:1 (mol:mol). Such a high drug-to-lipid ratio may be dependent on the number of metal ions inside the liposome and/or the nature of the complex formed.

Formation of a transition metal complex with the therapeutic agents (e.g., Cu(DDC)₂) may be rapid, occurring in minutes, or more gradual (e.g., Cu-CX5461). The complexation reaction rate may be temperature dependent. The rate of metal-drug complex formation may also be dependent on the rate at which the externally added therapeutic agent crosses the lipid bilayer of the liposome. As will be appreciated by those of skill in the art, these variables can be adjusted as desired to achieve a desired reaction rate for the complexation reaction.

In certain embodiments, it is not desirable to add an ionophore to a liposome bilayer after loading of a poorly soluble therapeutic agent in the liposome as the inclusion of such a component may aid in imposing a pH gradient across the bilayer. The ionophore facilitates the movement of two protons from the external buffer inside the liposome in exchange for one divalent cation, such as Mn²⁺, Cu²⁺, Mg²⁺ and Zn²⁺. Since loading as described herein is independent of a pH gradient, such ionophores may not be required to practice the invention. Indeed, the use of an ionophore can serve to reduce the internal transition metal concentration. Thus, according to one exemplary embodiment, the liposome does not comprise an ionophore used to establish a pH gradient across the bilayer of the liposome.

Without being limiting, for therapeutic agents whose solubility decreases in the presence of a metal ion, it has been found that the formation of the metal complex in the internal solution of the liposome appears to increase the solubility of the therapeutic agent in the internal solution. Without being limiting, an example of such a therapeutic agent is DDC. This therapeutic agent is insoluble in solution when complexed with a metal ion, but soluble in water. However, when complexed with metal in the internal solution of the liposome, precipitation does not appear to occur. In one embodiment, the drug-metal complex could potentially exceed its solubility relative to its solubility in free solution. The therapeutic agent-metal complex may also be present as a colloid in suspension. In another embodiment, the therapeutic agent is in a non-precipitated form within the internal solution of the liposome. Conversely, for therapeutic agents that are more soluble in the presence of a metal ion, the formation of a metal complex in the internal solution of the liposome may increase the solubility of the therapeutic agent in the internal solution.

Combinations of Therapeutic Agents

Advantageously, the method described herein can be used to load multiple therapeutic agents, either simultaneously or sequentially. Each of the therapeutic agents incorporated into the liposome can be loaded by the complexation method described herein. Moreover, the liposomes into which the therapeutic agents are loaded may themselves be prepared so that the internal solution comprises not only the metal ion but also a therapeutic agent. Loading of a therapeutic agent in this manner is often referred to as passive loading. The subsequent loading of the poorly soluble therapeutic agent which complexes with the metal in the preformed liposome (as described above) will result in encapsulation of two therapeutic agents, one of which is loaded passively and the other actively via complexation. Since the passively loaded therapeutic agent need not complex with metal ion to effect loading, this approach provides great flexibility in preparing liposome-encapsulated drug combinations for use to treat or prevent a disease of interest. A formulation of liposomes may also comprise two or more populations of liposomes (which entrap the same or different therapeutic agents), comprise different lipid formulations, or comprise different vesicle sizes. The combinations of therapeutic agents may be administered in order to achieve greater therapeutic efficacy, safety, prolonged drug release or targeting. For example, the two or more therapeutic agents may be loaded at a predetermined ratio that exhibits synergistic or additive effects as elucidated by the Chou-Talalay determination.

Examples of additional therapeutic agents that can be incorporated in a liposome in addition to the poorly soluble therapeutic agent loaded by metal complexation includes anthracyclines such as doxorubicin, daunorubicin, idarubicin, epirubicin and camptothecins such as topotecan, irinotecan, lurtotecan, 9-aminocamptothecin, 9-nitrocamptothecin and 10-hydroxycamptothecin.

According to one embodiment, therapeutic agents that can be encapsulated in a liposome in addition to the therapeutic agent loaded by metal complexation includes a second therapeutic agent in free form that becomes active in the presence of the metal ion. Examples of such drug combinations include co-encapsulation of metal-CQ and free DSF, the precursor of DDC. The DSF is metabolized for form DDC and DDC is then activated in the presence of a metal ion, such as copper, at the tumour site.

CX5461 Pharmaceutical Compositions

Embodiments of the invention also provide a pharmaceutical composition of metal complexed CX5461 for the treatment of disease including cancer. As set out above, CX5461 is presently in clinical trials as a cancer therapeutic, but has poor solubility at neutral pH. In order to overcome the low solubility at physiological pH, the drug can be dissolved in a solution having a pH of less than 4.5 or provided in the form of a slurry. However, these pH conditions are near the lowest that are tolerable for intravenous injection and could present potential inconsistencies in dosage due to the risk of precipitation upon introduction to physiological pH.

It has been discovered that the solubility of metal complexed CX5461 is greatly enhanced over CX5461 alone at physiological pH. The addition of metal to CX5461 resulted in activity that was similar to the low pH preparation of the metal-free drug. Solubility at this pH confers desirable pharmacokinetic properties, such as improved absorption and bioavailability as well as the ability to deliver higher dosages of CX5461.

Thus, according to certain embodiments of the invention, there is provided a pharmaceutical composition comprising CX5461 having the following Formula I:

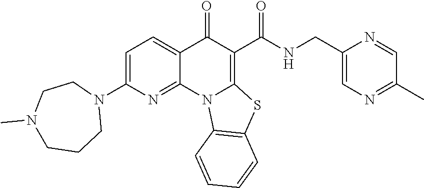

Formula I

wherein the CX5461 is complexed with a metal ion. Examples of suitable metal ions include transition metals or those of Group IIIb.

The pharmaceutical composition may comprise a pharmaceutically acceptable diluent or adjuvant. The pharmaceutical composition may comprise liposomes having encapsulated therein the CX5461 complexed with the copper or zinc. Alternatively, the pharmaceutical composition comprises CX5461 not encapsulated in a drug delivery vehicle such as the lipid-based nanoparticulate formulations described herein.

Administration

Embodiments of the invention also provide methods of administering the pharmaceutical composition comprising CX461 or liposomes to a mammal. The pharmaceutical composition may be administered to treat and/or prevent disease. The pharmaceutical composition will be administered at a dosage sufficient to treat or prevent the disease.

In one embodiment, the pharmaceutical compositions are administered parentally, i.e., intra-arterially, intravenously, subcutaneously or intramuscularly. In other embodiments, the pharmaceutical composition may be administered topically. In still further alternative embodiments the pharmaceutical composition may be administered orally. In a further embodiment, the pharmaceutical composition is for pulmonary administration by aerosol or powder dispersion.

The following examples are given for the purpose of illustration only and not by way of limitation on the scope of the invention.

### EXAMPLES

### Materials and Methods

Materials 1,2-distearoyl-sn-glycero-3-phosphocholine (DSPC), Cholesterol (chol) and (DSPE-PEG₂₀₀₀) were obtained from Avanti Polar Lipids (Alabaster, AL) and ³H-cholesteryl hexadecyl ether (3H-CHE) from PerkinElmer Life Sciences (Boston, Mass.). Pico-Fluor 40 scintillation cocktail was obtained from PerkinElmer Life Sciences (Woodbridge, ON, Canada). Disulfiram, Sodium Diethyldithiocarbamate trihydrate, Copper Sulfate, HEPES, Sephadex G-50, Clioquinol, Quercetin (Reagent grade) and all other chemicals were obtained from Sigma Aldrich. CX5461 was purchased from Selleck Chemicals.

Cytotoxicity Experiments

For studies with DDC, the cell lines U87, and A549 were obtained from ATCC, HBEpC (Human Bronchial Epithelial Cells) was obtained from Cell Applications (San Deigo, Calif.) and MDA-231-BR was from the NIH/NCI. The U251MG glioblastoma cell line (formerly known as U-373 MG) was originally obtained from American Type Culture Collection (Manassas, Va.) and was used for a maximum of fifteen passages. Subsequently, the U251MG was obtained from Sigma-Aldrich (product number 09063001). A microsatellite analysis was performed in order to compare these cells and the results indicated that the original cell line was derived from the Sigma-Aldrich sourced cells; however, the original line acquired deletions encompassing 21q21.1 and 21q22.3 suggesting chromosomal instability. Both cell lines are now being maintained as separate lines U251MG^{O} (original line) and U251MG^{SA} (Sigma-Aldrich). U87, U251MG^{O}, A549 and MDA231-BR cells were maintained in DMEM (Gibco) supplemented with 2 mM L-glutamine (Gibco) and 10% fetal bovine serum (Gibco). HBEpC were grown in bronchial/tracheal epithelial growth medium obtained from Cell Applications and were used for a maximum of three passages. All cells were maintained at 37° C. and 5% CO₂ The cells were seeded into 384 well plates and allowed to grow for 24 hrs and then treated as specified for 72 hours. To assess the cytotoxic effects of the indicated compounds in adherent cell lines, the cells were stained with Hoescht 33342 and ethidium homodimer I for total and dead cell counts, respectively. Twenty minutes later, the cells were imaged using an In Cell Analyzer 2200 and cell viability was measured based on viable nuclei count. For the suspension cell line MV-4-11, cells were incubated with the PrestoBlue reagent (Life Technologies) at 37° C. and 5% CO₂ for 1 hour, after which cell viability was evaluated based on metabolic activity as measured with the FLUOstar OPTIMA microplate reader (BMG Labtech).

Lipid-Based Nanoparticulate Preparation

Liposomes (80 nm) were prepared by extrusion and were composed of DSPC/Chol (55:45 mol ratio) or DSPC/Chol/DSPE-PEG₂₀₀₀ (50:45:5 mole ratio). Briefly, lipids were desiccated for 2 hours after removal from the freezer (−80° C.), weighed and dissolved in chloroform at the ratios indicated. The non-exchangeable and non-metabolizable lipid marker ³H-CHE was incorporated into the chloroform mixture. The chloroform was removed under a stream of nitrogen gas prior to being placed under high vacuum for at least 3 hrs to remove residual solvent. The resultant lipid film was hydrated (total lipid concentration of 50 mM) by adding unbuffered 300 mM CuSO₄ (pH 3.5) at 65° C. for at least 2 hours with frequent vortex mixing. Subsequently, the hydrated lipids underwent 5 freeze (in liquid nitrogen) and thaw (65° C. water bath) cycles. The hydrated lipids were then placed in an Extruder™ (Northern Lipids Inc.) and extruded through stacked 0.08 μm polycarbonate filters (Whatman® Nucleopore) 10 or 20 times. The size of the resulting liposomes was determined using quasi-electric light scattering (NanoBrook ZetaPALS Potential Analyzer). Prior to adding the specified copper-binding drug, unencapsulated CuSO₄ was removed by running the sample through a Sephadex G-50 column equilibrated with sucrose (300 mmol/L), HEPES (20 mmol/L) and EDTA (15 mmol) at pH 7.5 (SHE buffer). For studies with DDC, EDTA was subsequently removed by running the sample through a Sephadex G-50 column equilibrated with sucrose (300 mmol/L) and HEPES (20 mmol/L) (pH 7.5). The sample was subsequently concentrated to the desired lipid concentration using tangential flow dialysis.

Liposomal lipid concentration was determined by measuring ³H-CHE using liquid scintillation counting (Packard 1900TR Liquid Scintillation Analyzer). For studies with CX5461, the external SHE buffer was exchanged to 50 mM sodium phosphate, pH 3.5 via size exclusion chromatography (SEC) prior to drug loading.

Copper Complexation Reactions

Copper loaded-liposomes were mixed with DDC (4 or 25° C.), CQ (40° C.), Qu (50° C.) or CX5461 (60° C.) at the indicated compound-to-liposomal lipid ratio in the Sucrose/Hepes buffer (pH 7.4) and incubated over a 60-min time course. The reaction between the added compound and encapsulated copper to form a copper complex was detectable by eye as a change in the colour of the solution. Liposome and associated compound were separated from unassociated (free) compound using a Sephadex G-50 column equilibrated with SH buffer. The eluted liposome fractions (collected with the excluded volume of the column) were analyzed for copper, compound (as the copper complex or after dissociation of the bound copper) and liposomal lipid concentrations. Lipid concentrations were measured by assaying for [3H]-CHE by liquid scintillation counting (Packard 1900TR Liquid Scintillation Analyzer) where 20 μL of eluted liposome sample was dissolved in 5 mL Pico-Fluor Plus (Perkin Elmer). For the spectrophotometric assay, samples were diluted into 1 mL methanol for Cu(DDC)₂ and Cu(CQ)₂ and absorbance was measured at 435 nm (1-10 μg/mL) or 275 nm (0.25-2.5 μg/mL), respectively. CuQu and CuCX5461 were dissolved in 1 mL of 3% acetic acid in methanol and Qu and CX5461 were measured by assessing absorbance at 372 nm (1-10 μg/mL) or 288 nm (1-10 μg/mL), respectively. Copper was measured using atomic absorption spectrophotomer (AAnalyst600, Perkin Elmer). The Cu-containing liposomes were diluted in 10 mLs of 0.1% HNO₃.

A copper (Cu²⁺) standard curve was generated using Cu²⁺ (from 0-100 ng/mL) in 2% nitric acid (Sigma Aldridge).

Characterization of Liposomes

All formulations were characterized for surface charge, size and polydispersity. Samples were diluted to 1-5 mM in filtered 0.9% NaCl or SH buffer for size and polydispersity analysis. Surface charge measurements were performed in a 1 mM KCl solution. Further analysis of the Cu(DDC)₂ formulations was performed by cyro-electron microscopy (CEM). CEM analysis was performed using a Zeiss Libra 120 transmission electron microscope at the University of Uppsala, Sweden. Briefly, liposomes were prepared as described above containing either Cu(SO₄)₂ or Cu(DDC)₂ with SH buffer at pH 7.4. In a controlled chamber for humidity and temperature (25° C.) samples of 1-2 μL of the sample were deposited on copper grids coated with a cellulose acetate butyrate polymer having holes formed therethrough. Excess liquid was blotted away carefully with filter paper and then samples were quickly vitrified by plunging into liquid ethane. The samples were then transferred to liquid nitrogen to maintain the temperature below 108 K, which minimizes formation of ice crystals. Images were taken in a zero-loss bright-field mode and an accelerating voltage=80 kV.

Parenteral (Intravenous) Administration of Formulations

Female CD-1 mice were given bolus tail vein intravenous injections of Cu(DDC)₂ (15 mg/kg, drug-to-lipid ratio 0.2 mol:mol), CuCQ (30 mg/kg, drug-to-lipid ratio 0.2 mol:mol), CuQu (70 mg/kg, drug-to-lipid ratio 0.2 mol:mol), or CuCX5461 (50 mg/kg, drug-to-lipid ratio 0.2 mol:mol). All formulations were prepared using DSPC:Chol (55:45) liposomes with encapsulated 300 mM copper sulfate as described above. To define tolerability of the formulations, mice (n=3) were given the drug at a specified dose and monitored for changes in body weight, appearance and behaviour. Health assessment was completed using a standard operating procedure (SOP), approved by the Institutional Animal Care Committee. The health of the animals was measured over a 14 day period after administration and a full necropsy was performed at that time to assess for changes in tissue/organ appearance. Once a safe dose was defined, pharmacokinetic studies was completed where blood was collected by cardiac puncture in mice terminated at 1, 4, 8 and 24 hours (n=4 per time point) by isoflurane followed by CO₂ asphyxiation. Blood was placed into EDTA coated tubes and stored at 4° C. until they were centrifuged at 2500 rpm for 15 min at 4° C. in a Beckman Coulter Allegra X-15R centrifuge. Plasma was collected and stored at −80° C. until they were assayed by AAS (see above) for copper, liposomal lipid (see above) or compound as described below.

Quantification of CuDDC₂ (by AAS) and Clioquinol, Quercetin, and CX5461 (by HPLC)

Cu(DDC)₂ was measured by using Cu as a surrogate marker. Samples were diluted in 0.1% HNO₃ and subsequently the Cu concentration was measured using AAS (AAnalyst600, Perkin Elmer) as described above. Plasma Cu was corrected using untreated CD-1 mouse plasma as a blank. An HPLC assay for Cu(DDC)₂ was developed, but the limits of detection were too low to provide meaningful data in the pharmacokinetic studies. All other compounds were measured using HPLC as summarized below using a Waters Alliance HPLC Module 2695 and photodiode array detector model 996 and Empower 2 Software. Clioquinol was measured at 254 nm following separation on a X-terra C18 column (3.5 μm, 3.0×150 mm) using a 1:1 mobile phase of water (pH 3 phosphoric acid) and acetonitrile. A 30 μL sample volume was injected, the flow rate was 1 mL/min and column temperature was set at 55° C. Pyrrolidine diethyldithiocarbamate was added to samples and standards at an excess of 3 mol equivalents prior to injection to ensure dissociation of CQ from Cu. Quercetin was measured at 368 nm following separation on a symmetry C18 column (3.5 μm, 3.0×150 mm) using a mobile phase of 0.1% TFA in water and acetonitrile (2.3:1). A 25 μL sample volume was injected, the flow rate was set at 1 mL/min and the column temperature was 30° C. Samples and standards were prepared in acidified methanol so as to dissociate the CuQu complex prior to HPLC analysis. Similarly, the quantification of CX5461 was performed in acidified methanol to dissociate the complex and CX5461 was measured at 300 nm following separation on a Luna C18 column (5 μm, 4.6×150 mm). The mobile phase contained a 1:1.2 mixture of 0.1% TFA in water and 0.1% TFA in methanol. A 5 μL sample volume was injected, the flow rate was set at 1 mL/min and the column temperature was 35° C.

### Results

### Example 1

### A Metal Ion can Increase the Cytoxicity of a Poorly Soluble Drug

This example shows that the cytotoxic activity of diethyldithiocarbmamate (DDC) can be increased in the presence of a metal ion. In this example, the metal ion was Cu2+.

Disulfiram (DSF) is metabolized to diethyldithiocarbmamate (DDC) (FIG. 1A) and DDC is a copper chelator. As shown in FIG. 1(A), the precursor molecule, disulfiram (DSF) is metabolized to diethyldithiocarbamate (DDC) through cleavage of a sulfur-sulfur bond. This produces two negatively charged molecules of DDC, in which the negative charge is de-localized over two sulfur atoms. The two molecules of DDC can then complex with copper (Cu²⁺) through coordination with the negatively charged sulfur atoms. Unlike DDC, Cu(DDC)₂ is highly insoluble in water.

The cytotoxic activity of DSF when added to cancer cells is increased in the presence of the metal. As shown in FIG. 1B, the IC₅₀ of DSF against U87 glioblastoma cells is >10 μM in the absence of copper. In the presence of copper there is a substantial shift (2-orders of magnitude) in cytotoxicity when copper was added with DSF at a 1:1 molar ratio. DSF is unable to interact with copper, thus the activity of DSF depends on its degradation to DDC. As shown in FIG. 1C, the activity of DDC in the absence of copper is also >10 μM and in the presence of copper (2:1 molar ratio of DDC to copper) was approximately 220 nM. Similar results were obtained in 4 other cell lines where the IC₅₀ of copper+DDC was 345, 329 and 880 nM when used against U251 (glioblastoma line), MDA-231BR (a triple negative breast cancer line selected for its propensity to metastasize to the brain) and A549 (lung cancer line) cells, respectively. (See FIG. 1D). DDC as well as Cu(DDC)₂ exhibited little activity when added to normal human bronchial epithelial cells HBEpC, suggesting specificity of Cu(DDC)₂ against cancer cells.

Thus, the results above support that the utilization of DSF as an anticancer drug should focus on Cu(DDC)₂. However, Cu(DDC)₂ is almost completely insoluble in aqueous solution (FIG. 1E). As discussed below, the inventors discovered that this limitation to its therapeutic potential as a cancer drug can be overcome by incorporation in liposomes.

Cytotoxicity results were obtained with an IN CELL™ Analyzer in U87 glioblastoma cells. Cell viability was assessed based on detection of plasma membrane integrity 72 hours following treatment. Total and dead cell counts were determined using Hoeschst 33342 and ethidium homodimer staining.

### Example 2

### Overview of the Metal-Complex Based Loading Method

DDC-copper complex formation was confirmed by UV spectroscopy. Both CuSO₄-liposomes and Cu(DDC)₂-liposomes (5 mM) were dissolved in methanol and subsequently measured on a UV-Vis spectrophotometer. Drug-metal complex formation can be seen through a shift in absorbance at 435 nm.

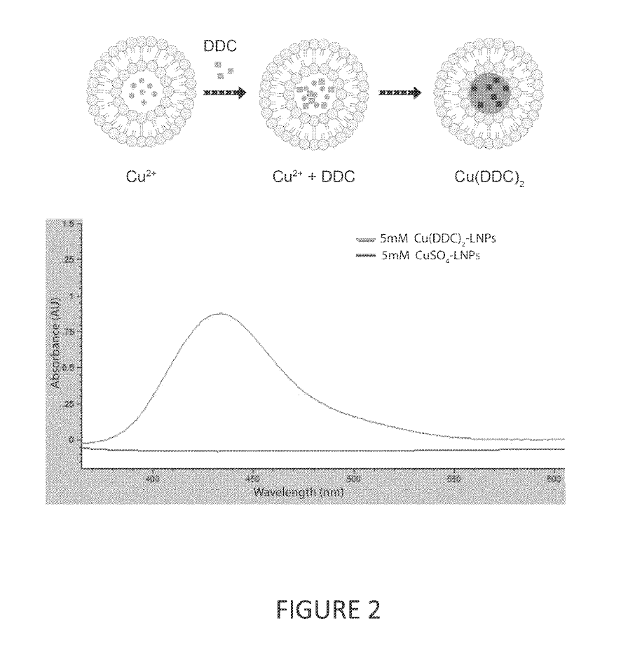

The scheme for loading a drug into a liposome that is poorly soluble in a copper-containing solution is depicted graphically in FIG. 2. Copper (Cu²⁺)-containing liposomes were prepared as described above. The internal solution contains unbuffered CuSO₄ (pH 3.5). After preparation, the Cu²⁺ liposomes are mixed with the therapeutic agent, which in this example is DDC. The DDC crosses the lipid bilayer and the resulting liposomes are produced with the Cu-complex suspended inside.

### Example 3

### The Insolubility of Poorly Soluble Drugs can be Overcome by Encapsulation in Metal Ion-Containing Liposomes

As noted, therapeutic agents that are insoluble in aqueous solution (<1 mg/mL) are not suitable for parenteral or oral administration. However, as demonstrated below, the insolubility of Cu(DDC)₂ can be overcome by incorporation in liposomes.

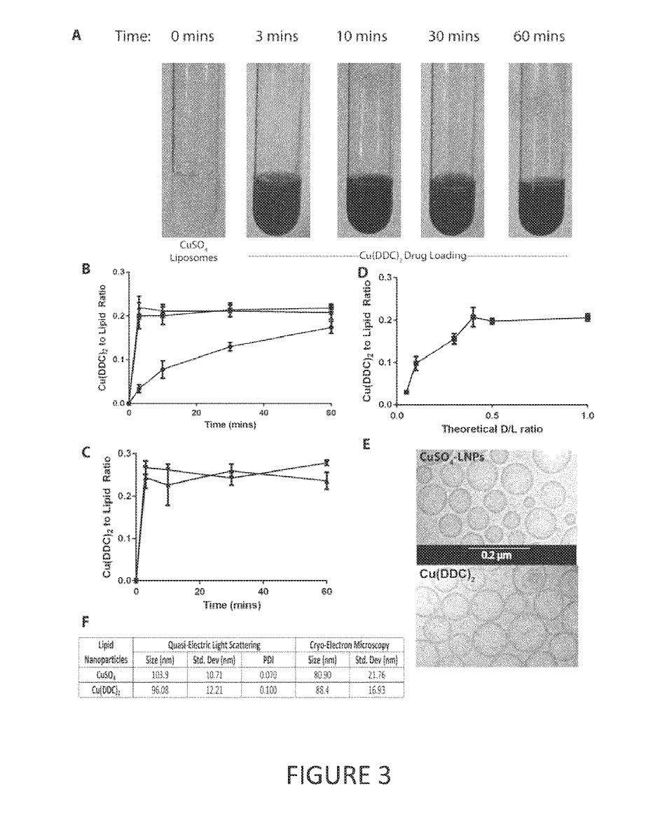

As illustrated in FIG. 3, within minutes after the addition of DDC to preformed liposomes comprising encapsulated copper, there is a color change observed visually that is indicative of Cu(DDC)₂ complex formation (FIG. 3A).

Drug loading time course studies were next conducted with DSPC/Chol (55:45, molar ratio) liposomes prepared as described above. The rate of Cu(DDC)₂ formation inside the liposome was quantified by separating liposome-associated Cu(DDC)₂ from unassociated DDC and then assaying for Cu(DDC)₂ using UV-Vis spectroscopy and lipid was measured using scintillation counting.

As shown in FIG. 3B, Cu(DDC)₂ association is rapid when DDC is added to copper-containing liposomes at 20° C. (room temperature) and at 40° C., where the maximum Cu(DDC)₂ to lipid ratio of 0.2 (mol ratio) is achieved within 3 minutes. If the temperature is decreased to 4° C., the Cu(DDC)₂ to lipid ratio of 0.2 (mol ratio) is achieved at 60 minutes.

Notably, the movement of DDC from the external media to the copper-containing liposomal core is not affected by pH. As shown in FIG. 3C, when the external pH is adjusted to 3.5 the loading rate is comparable to that observed at pH 7.4. To determine the maximum Cu(DDC)₂ to lipid ratio that can be achieved when using liposomes prepared in 300 mM copper sulfate, the amount of external DDC was titrated from 0.04 to 0.40 (moles DDC to moles liposomal lipid) and the results suggest (FIG. 3D) that the maximum Cu(DDC)₂ to lipid ratio achievable under these condition was 0.2 (mol:mol). This was achieved when the initial DDC to liposomal lipid ratio was 0.4 (mol:mol).

As indicated, Cu(DDC)₂ forms an insoluble precipitate in solution and it was possible that formation of Cu(DDC)₂ inside the liposomes may have also caused formation of a precipitate within the liposomal core. To evaluate this, the liposomes were visualized by cryo-electron microscopy (FIG. 3E). The results illustrate two notable observations: (1) the Cu(DDC)₂ liposomes exhibited a mean particle size that was comparable to that observed with the copper-containing liposomes before addition of DDC, and (2) the formation of Cu(DDC)₂ inside the liposomes did not result in the formation of an electron dense core suggestive of Cu(DDC)₂ precipitation. It should be noted that the liposome size estimated by Cryo-electron microscopy analysis was comparable to that determined by quasi-electric light scattering (FIG. 3F).

### Example 4

### Incorporation of PEG-DSPE can Increase the Amount of Encapsulated Metal

The influence of the incorporation of polyethylene glycol (PEG₂₀₀₀) modified DSPE on liposomal lipid composition was considered. PEG₂₀₀₀-DSPE is a negatively charged lipid and its inclusion in the liposome bilayer could increase the amount of encapsulated copper when preparing the liposomes. Moreover, PEG₂₀₀₀-DSPE prevents surface-surface associations that can influence liposome-liposome aggregation and liposome-cell interactions which, in turn, affect elimination rates in vivo.

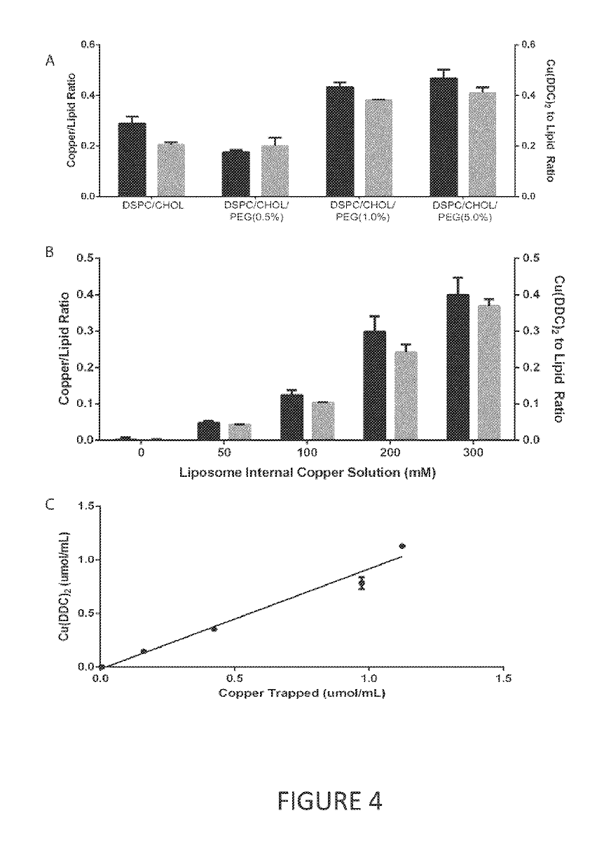

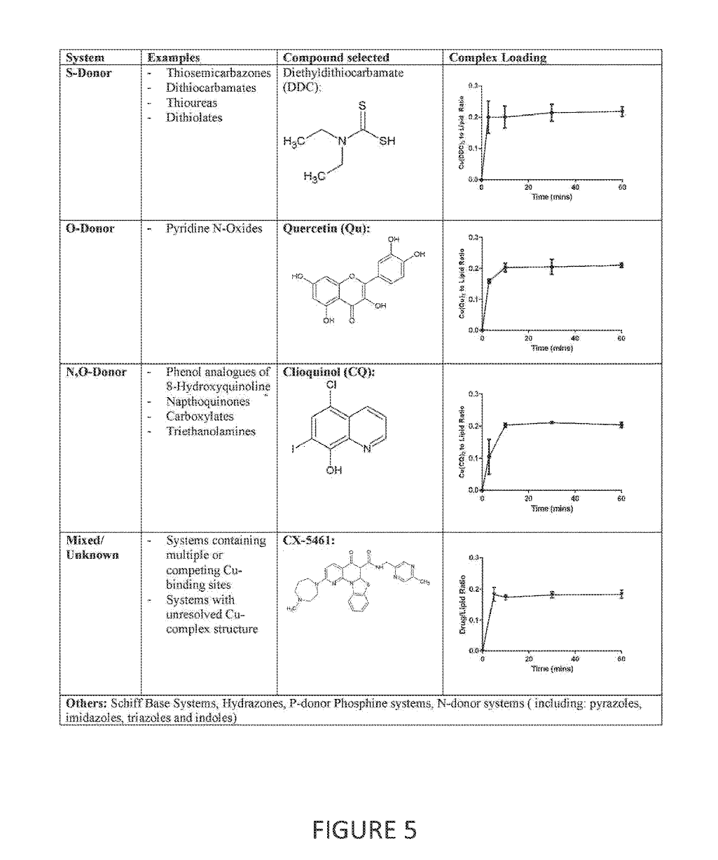

When PEG₂₀₀₀-DSPE was added to the base lipid formulation of DSPC:CHOL (55:45, mole ratio) ranging from 0.5 to 5% (based on reductions of DSPC content) the maximum amount of liposome-associated Cu(DDC)₂, as measured by the Cu(DDC)₂ to liposomal lipid ratio, increased from 0.2 to 0.4 (FIG. 4A, black bars). When analyzing the amount of copper associated with these liposomes (gray bars) it was clear that the Cu(DDC)₂ to liposomal lipid ratio was related to the amount of copper retained in the liposomes. The addition of PEG₂₀₀₀-DSPE increased copper encapsulation. Without being bound by theory, this is likely due to the introduction of an anionic change that enhances liposome trapped volume.

The DSPC/CHOL/DSPE-PEG₂₀₀₀ (50/45/5 mol ratio) was selected to establish the relationship between the amount of encapsulated copper and final Cu(DDC)₂ to liposomal lipid ratio. These liposomes were prepared using copper sulfate solutions with copper concentrations ranging from 0 to 300 mM. The osmolarity (˜300 mOs/kg) of these solutions was balanced with MgSO₄.

These liposomes were analyzed for copper content prior to DDC addition and after addition of DDC in excess (>2-fold molar excess to the measured liposome associated copper for liposomes prepared in the 300 mM copper sulfate solution). The results (FIG. 4B and 4C) are consistent with the data in FIG. 4A. That is, the Cu(DDC)₂ to liposomal lipid ratio achieved was directly proportional to the amount of copper retained in the liposomes. A plot of encapsulated copper vs encapsulated Cu(DDC)₂ demonstrated a linear regression fit of R²=0.9754. This is consistent with a 1:1 mol ratio between copper and Cu(DDC)₂ or a 1:2 ratio of copper to DDC.

Copper was measured using atomic absorption spectroscopy, Cu(DDC)₂ was measured using UV-Vis spectroscopy and lipid was measured using scintillation counting.

### Example 5

### Other Donor Systems can be Used in Copper(II)-Complex Loading

The results summarized above describe an injectable liposome formulation of Cu(DDC)₂. However, the foregoing liposomal formulations are compatible with other copper-binding drugs and drug candidates. To assess the breath of this approach, other therapeutic agents that encompass a range of functional group donor types have been evaluated. In particular, each agent was assessed for its loading characteristics when added to liposomes comprising copper.

These agents are summarized in FIG. 5 and include, but are not limited to, S-Donor, O-Donor and N,O-Donor systems. Examples tested, in addition to DDC (an S-Donor), include Quercetin (Qu) (an O-Donor), Clioquinol (CQ) (an N, O donor) as well as a compound, CX5461, previously not identified as a copper complexing agent. The indicated therapeutic agents are poorly soluble in aqueous solutions at pH 7.4 and can be encapsulated when added to pre-formed liposomes DSPC/CHOL (55:45 molar ratio) prepared with encapsulated copper. The therapeutic agents, Qu and Clioquinol, were added in a solid/powdered form. CX5461 was prepared as a metastable solution in low pH (3.5) phosphate buffer.

As noted in FIG. 5 (far right column) all formulations could be designed to achieve a final Cu-complexed drug to liposomal lipid molar ratio of 0.2. In each example, loading was rapid at the optimal temperature. The Cu(DDC)₂formation rate was optimal at 25° C., Cu(CQ)₂ formation was optimal at 40° C., the Cu(Qu) and Cu(CX5461) formations were optimal at 50° C. and 60° C., respectively. It will be appreciated that an optimal loading temperature for a drug in question can readily be determined by a person of ordinary skill in the art.

### Example 6

### Copper Forms a Complex with CX5461

As noted above, the drug CX5461 has not previously been identified as a copper complexing agent. The UV-Vis, NMR and EPR spectra presented below, however, suggest that CX5461 complexes with copper. Proton NMR results are also presented with zinc.

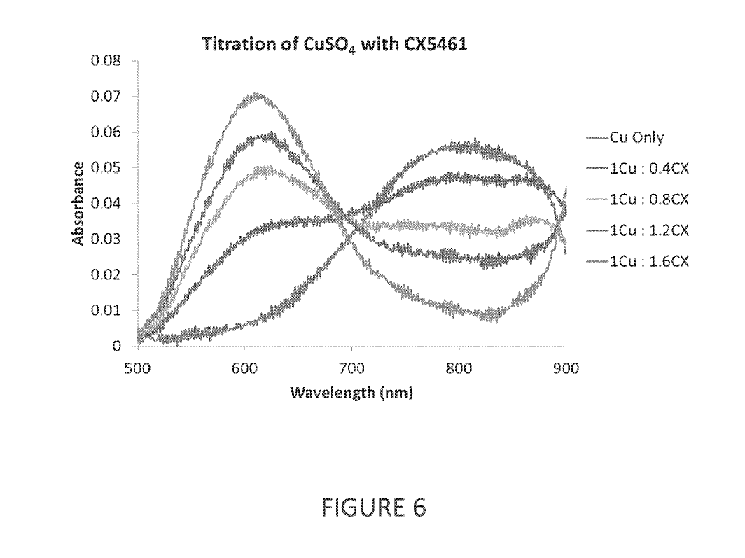

UV-Vis titrations were performed by incrementally adding CX5461 to a 5 mM solution of CuSO₄. The diagnostic metal absorption bands of a Cu-CX5461 complex were monitored in the UV-Vis spectrum. The results are shown in FIG. 6. The initial absorbance from solvated Cu²⁺ (λₘₐₓ=800 nm and ε=12 M⁻¹cm⁻¹) was steadily replaced by a new absorbance λₘₐₓ=620 nm (E 20 M⁻¹cm⁻¹). The higher energy λₘₐₓ and increased extinction coefficient indicate an increase in the d-orbital splitting (Δ) of the copper center. This correlates with copper coordination to a stronger field ligand, such as the aromatic nitrogens of CX5461.

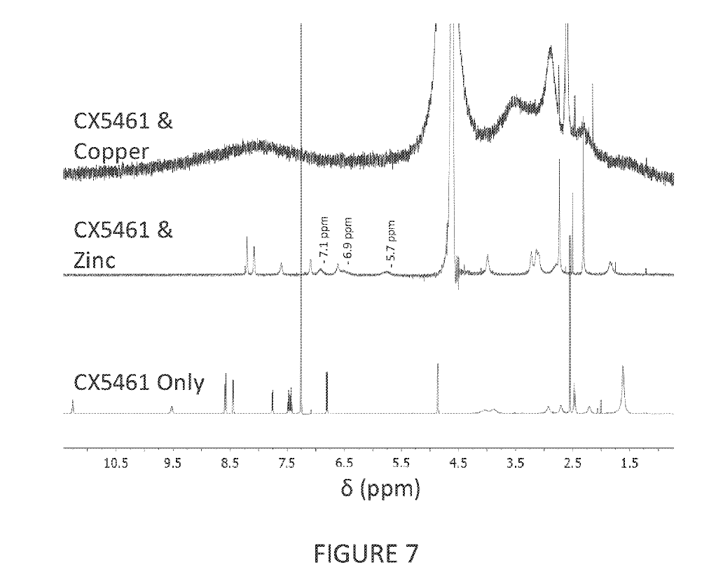

The proton NMR spectra of CX5461 alone were compared with the NMR spectra of CX5461 in combination with copper or CX5461 in combination with zinc. The results are shown in FIG. 7. As shown in the top portion of FIG. 7, characteristic paramagnetic broadening of ¹HNMR signals were observed due to the Cu(II) interacting with CX5461. The middle spectra of the figure indicate that when CX5461 is incubated with zinc, a broadening of the three indicated signals was observed, demonstrating that the pyridine of CX5461 is a likely location of metal coordination. The ¹HNMR spectra of CX5461 in chloroform was used (bottom of spectra) to demonstrate the changes upon incubation with metal salts.

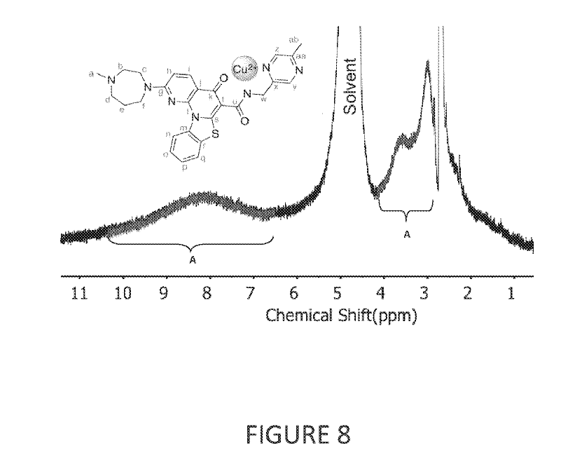

FIG. 8 is the proposed structure of Cu-CX5461. The sample tested was a 10 mM solution of zinc (II) chloride with 5 mM of CX5461 in D₂O at pD 6. 1D and 2D NMR analyses indicate that carbons x and z shifted downfield (>1 ppm) while carbons y and aa shifted upfield (>1 ppm). These results suggest that there is a binding pocket formed for the metal ion by the pyrazine nitrogen with two other donor atoms (N, 0) as indicated in the illustration.

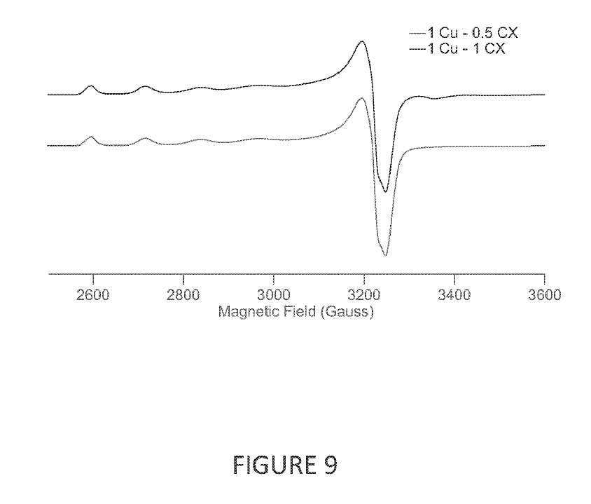

FIG. 9 is the Cu electron paramagnetic resonance (EPR) spectra of CuSO₄ in combination with CX5461. A change in the primary coordination sphere of CuSO₄ was observed upon the addition of increasing amounts of CX5461.

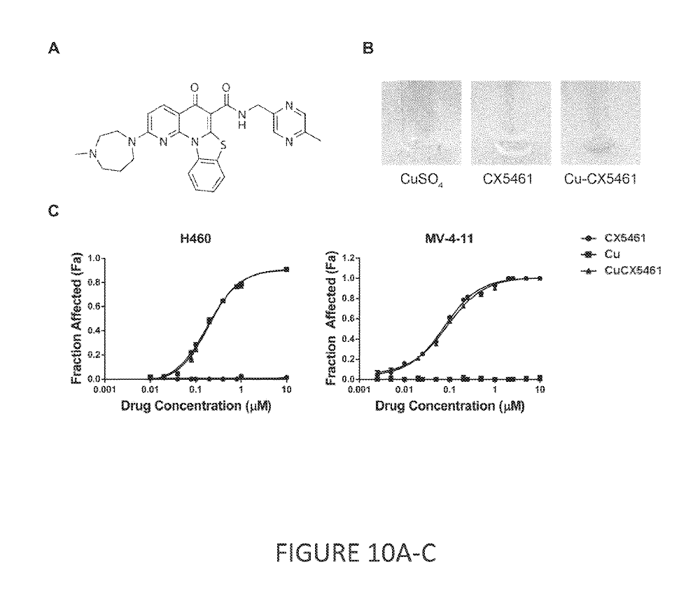

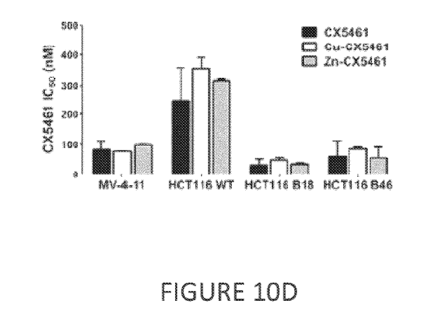

The formation of a Cu-CX5461 complex can also be identified visually by a colour change in solution. As shown in FIG. 10B, at equal concentrations, copper sulfate (CuSO₄) and CX5461 dissolved in NaH₂PO₄ are colourless solutions. When copper sulfate and CX5461 are combined, however, the solution becomes blue. As can be seen in FIG. 10B, the rightmost test-tube containing copper and CX5461 is darker in colour than the test-tubes containing copper sulfate or the drug alone.

### Example 7

### The Cytotoxicity of CX5461 in the Presence and Absence of Copper

The cytotoxicity of the drug CX5461 was tested in a 72-hour cytotoxicity assay as described above. The cytotoxicity of the drug CX5461 was tested in a 72-hour cytotoxicity assay as described above. For CX5461, the presence of equimolar copper does not alter the anti-cancer activity of CX5461 in H460 (non-small cell lung cancer) and MV-4-11 (biphenotypic B-myelomonocytic leukemia). The results are presented in FIG. 10C.

FIG. 10D shows the IC₅₀ (nm) values for CX5461 as measured in MV-4-11, HCT116WT, HCT116B18 and HCT116B46 cells. The combination of CuSO₄ or ZnSO₄ with CX5461, dissolution at pH 7.4, resulted in activity that was statistically similar to the low pH preparation of a metal-free compound. Thus, metal coordination enhances the solubility of CX5461 in aqueous solution, enabling low nM cytotoxicity to be achieved via dissolution at physiological pH.

### Example 8

### Encapsulation of CX5461 in Liposomes Using a Metal as the Driving Force

This example demonstrates that CX5461 can be encapsulated into DSPC/Chol (55:45, mol:mol) liposomes using a metal as a driving force and that the resultant liposomes were stable for at least 3 weeks. Liposomes containing encapsulated copper were prepared as described above and the external solution was exchanged with 50 mM sodium phosphate buffer, pH 3.5.

The drug CX5461 dissolved in sodium phosphate at pH 3.5 was loaded into copper-containing liposomes at different temperatures. The formulation was then cooled to room temperature. The external buffer was subsequently exchanged to HBS (20 mM HEPES, 150 mM NaCl, pH 7.4) via SEC and the final formulation was concentrated to the desired concentration using tangential flow filtration. The formulation was characterized based on size and polydispersity using a ZetaPALS particle sizer (Brookhaven Instruments Corp., Holtsville, N.Y.). Drug concentration and lipid concentration was determined via UV-Visible Spectroscopy at 288 nm and liquid scintillation counting using an Agilent 8453 UV-visible Spectrophotometer and L56500 Multipurpose Scintillation Counter.

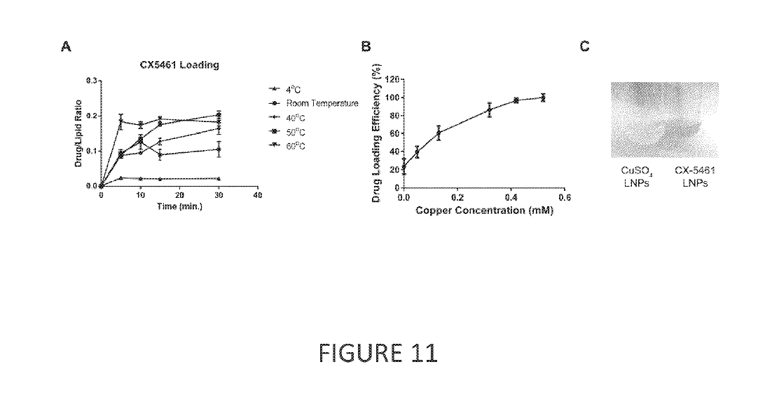

As shown, the drug-to-lipid ratio, a measure of the amount of CX5461 encapsulated into the liposomes, increased in a time and temperature-dependent manner (FIG. 11A). The loading efficiency is also dependent on the amount of copper present (FIG. 11B). Upon loading with CX5461, the liposome preparations became blue, indicating the formation of a Cu-CX5461 complex within the liposomes. These results are shown in FIG. 11C, which indicates that CuSO₄-containing LNPs (liposomes containing copper) without encapsulated drug were colourless and CX5461 LNPs (liposomes with copper and CX5461) were darker in colour.

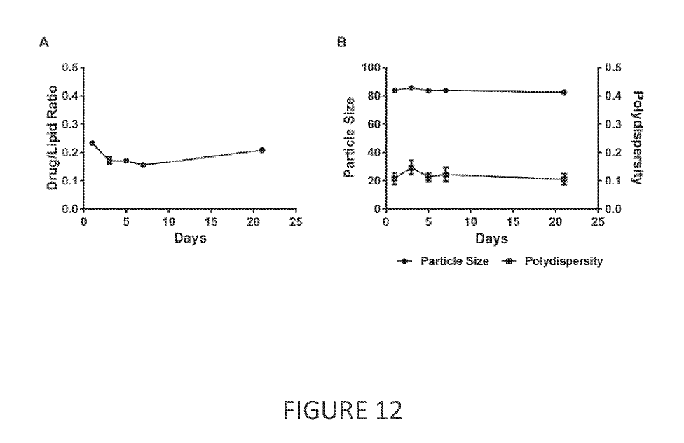

The stability of the drug loaded liposomes is shown in FIG. 12. The drug-to-lipid ratio (D/L; FIG. 12A), particle size, and polydispersity (FIG. 12B) of the liposomes were determined on days 1, 3, 5, 7, and 21, with day 1 being the day that the liposome was prepared. As demonstrated in the plots, the D/L ratio was maintained in the range of 0.15 to 0.2 (FIG. 12A). There was no significant change in the average particle size (approximately 83 nm) and the particles appeared to stay uniformly distributed with a polydispersity value of approximately 0.1 (FIG. 12B).

### Example 9

### The Encapsulation of CX5461 in Metal-Containing Liposomes can Enhance the Pharmacokinetics (PK) Profile and In Vivo Activity of the Agent

Metal complexed CX5461 encapsulated in liposomes displayed enhanced pharmacokinetics profiles and in vivo activity following parenteral administration.

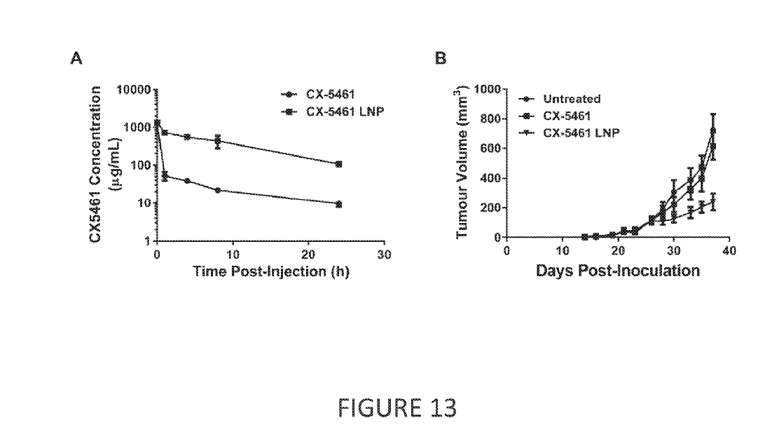

More specifically, FIG. 13 shows that CX5461 encapsulated in copper-containing liposomes enhances the pharmacokinetics (PK) profile and in vivo activity of CX5461. As shown in FIG. 13A, while 96% of the compound in the free form is removed from circulation within 1 hour of injection, more than 60% of the compound is still detected in the plasma when CX5461 was administered in liposomes co-encapsulated with copper.

In a xenograft model of MV-4-11, mice were inoculated with 1×10⁶ cells and treated with either free CX5461 or CX5461 LNP at 30 mg/kg (Q4Dx3) when the tumours were established (100-150 mm³). The tumour volumes shown in FIG. 13B indicate a significant delay in tumour growth when the mice were treated with the liposomal formulation (data plotted as mean ±SEM).

### Example 10

### Solubility of Quercetin in Water and an Aqueous Buffer

Quercetin is another therapeutic agent that has limited clinical usefulness but has low solubility in aqueous solution. As such, there is a need to improve the solubility of quercetin in order to realize its therapeutic potential.

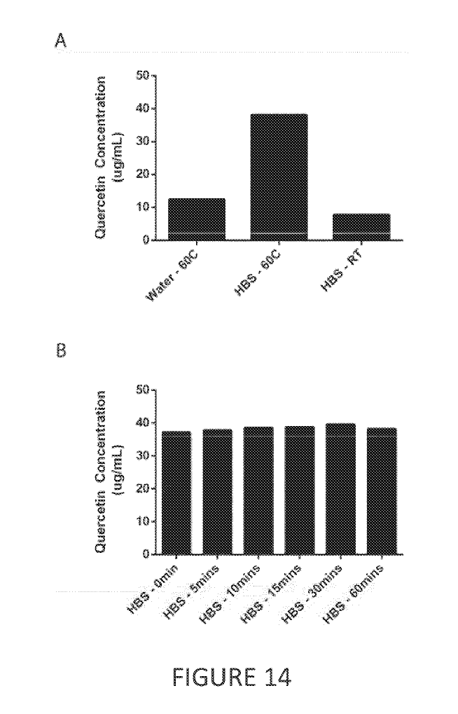

It was confirmed that quercetin exhibits limited solubility in water even when incubated at 60° C. (solubility 12.33 μg/mL at 60° C.). Solubility was increased in a balanced buffered solution (HBS) at room temperature (7.78 μg/mL) and at 60° C. (38 μg/mL). A supersaturated solution of quercetin-HBS remained stable over a one-hour period once removed from heat. The results are shown in FIG. 14.

### Example 11

### Quercetin Chemical Structure and Characterization of Copper-Based Loading Properties

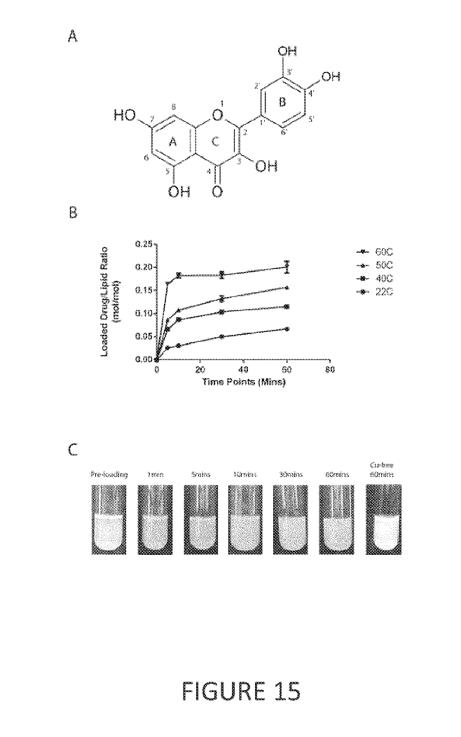

The structure of quercetin is shown in FIG. 15A. Quercetin is a triple-ringed flavonoid with capacity to chelate copper at three groups: 3′4′-dihydroxy group on the B ring, 3-hydroxy and 4-carbonyl group in the C ring, and the 5-hydroxy and 4-carbonyl group spans across the A and C rings (FIG. 15A).

Quercetin was loaded into 300 mM copper sulfate liposomes (55:45 molar ratio) in HEPES buffer saline (HBS) pH 7.4 at different temperatures (22° C., 40° C., 50° C. and 60° C., FIG. 15B). At 60 minutes, maximum loading of 0.2 mol/mol drug-to-lipid ratio was achieved at 60° C. and drug-to-lipid ratios of 0.16, 0.12 and 0.07 were reached at 50° C., 40° C., 50° C. and 60° C. (FIG. 15B). At all tested temperatures, maximal loading was achieved in 60 minutes. Colorimetric change from white to a yellow solution was also evident when copper liposomes were added to quercetin powder. As can be seen in FIG. 15C, the contents of the test-tubes are more darkened in colour moving from left to right as loading proceeds. The far right copper-free test-tube is white.

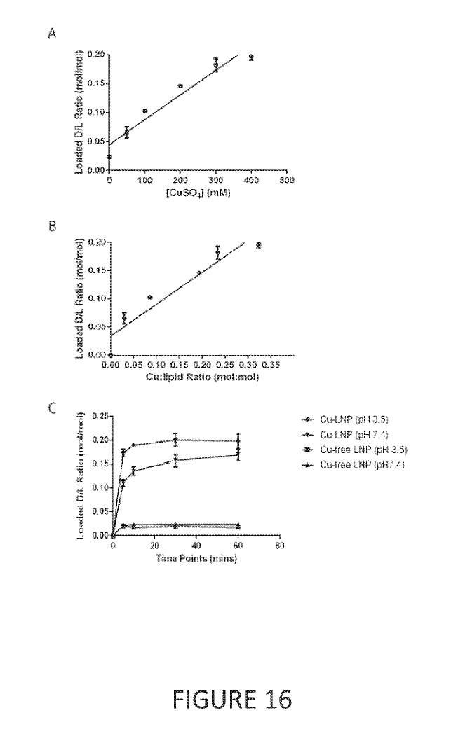

To examine the role of copper in efficient quercetin encapsulation, quercetin was loaded into liposomes containing various concentrations of CuSO₄ (50, 100, 200, 300 and 400 mM). As shown in FIG. 16A, the drug-to-lipid ratios of quercetin increased with increasing CuSO₄ concentrations. Further, there was no copper leakage during quercetin loading as the copper-to-lipid ratios (mol/mol) were similar for all CuSO₄ concentrations before and after loading. A plot of the post-loading drug-to-lipid ratio versus copper-to-lipid ratio revealed a linear relationship with a slop of 0.57, suggesting that a 1:2 (Q:Cu) complex was formed (FIG. 16B).

### Example 12

### Encapsulation of Quercetin into Liposomes is Metal-Dependent and is not Influenced by a pH Gradient

To further investigate whether the encapsulation of quercetin into liposomes was metal-dependent and/or pH gradient mediated, loading of quercetin into copper-containing and copper-free liposomes in the presence or absence of pH gradients were examined (FIG. 16C). Since a neutral pH could not be achieved with 300 mM CuSO₄ without precipitation, 100 mM copper gluconate was used to test whether the pH gradient across the liposome membrane was important for quercetin loading. Copper-free liposome controls were prepared using 300 mM citric acid (pH 3.7) and SH buffer (pH 7.4). In all cases, the external buffer was exchanged to HBS (pH 7.4). As shown in FIG. 16C, while pH gradients do not influence loading of quercetin into liposomes, copper appears essential for efficient loading. Specifically, drug-to-lipid ratios (mol:mol) were similar with and without a transmembrane pH gradient (0.19 for pH 3.5 and 0.17 for pH 7.4) in the presence of copper. However, without copper, quercetin loading was inefficient with drug-to-lipid ratios of 0.018 and 0.024 for SH (pH 7.4) and citric acid (pH 3.5) liposomes, respectively (FIG. 16C).

### Example 13

### Quercetin Loading into Liposomes Comprising CuSO₄ and Copper Gluconate in the Internal Solution

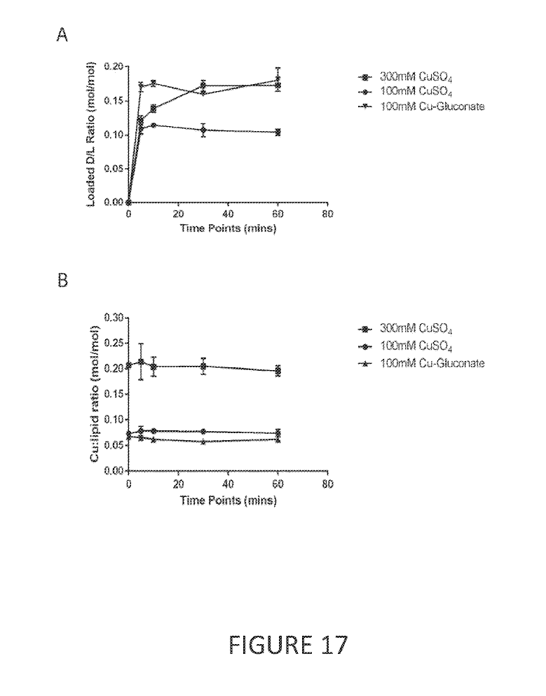

Time course loading studies were conducted using 100 mM copper gluconate (CuG), 100 mM CuSO₄ and 300 mM CuSO₄ as the buffers. As shown in FIG. 17A, similar drug-to-lipid ratios of 0.18 mol/mol and 0.17 mol/mol between 100 mM copper gluconate and 300 mM CuSO₄ liposomes, respectively, were evident. However, the drug-to-lipid ratio of 100 mM copper gluconate was almost double that compared to the use of 100 mM CuSO₄ (0.18 versus 0.10 mol/mol, respectively) (FIG. 17A).

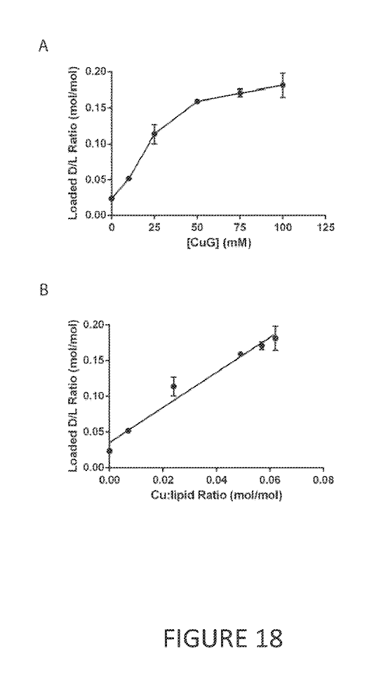

The amount of loaded copper in the liposomes was also compared with the amount of copper in the rehydration buffer. As shown in FIG. 17B, copper in liposomes rehydrated in 100 mM copper gluconate was one third of that in liposomes rehydrated with CuSO₄ (FIG. 17B). A positive correlation of quercetin loading with increasing concentrations of copper gluconate (CuG) was also found (FIG. 18A). The drug-to-lipid ratio versus copper-to-lipid ratio plot revealed a linear relationship with a slope of 2.55, which suggests the formation of a 2:1 (Q:Cu) complex (FIG. 18B).

### Example 14

### Copper Gluconate and Copper Sulfate Complex Formation with Quercetin

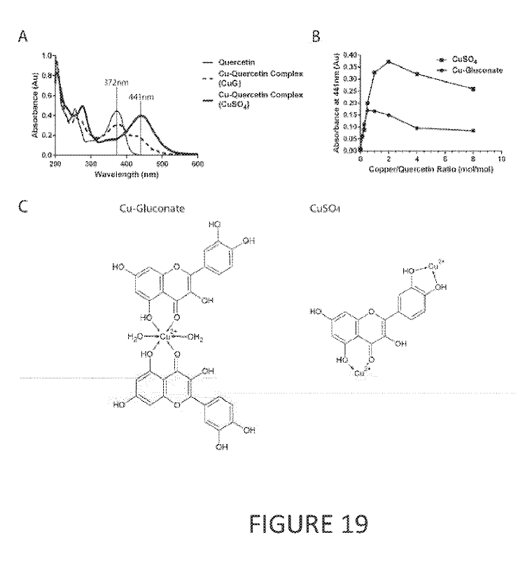

To examine whether different complexes were formed when quercetin interacts with copper sulfate and copper gluconate, UV absorption spectrophotometry was utilized. As shown in FIG. 19A, quercetin alone exhibits an absorption UV peak at 372 nm while complexation with copper shifts the maximal absorbance to 441 nm. Complexation with copper sulfate resulted in a more distinct peak than copper gluconate (FIG. 19A). A titration assay using varying concentrations of copper to a fixed quercetin concentration revealed that a maximum UV absorbance of 441 nm was achieved at cooper/quercetin ratio (mol/mol) of 2 for copper sulfate and 0.5 for copper gluconate (FIG. 19B). Without being bound by theory, these findings support that quercetin forms a 2:1 (quercetin:copper) complex in the presence of copper gluconate and a 1:2 (quercetin:copper) complex in the presence of copper sulfate (FIG. 19C).

### Example 15

### Stability of Quercetin-Loaded Liposomes Incubated in Fetal Bovine Serume (FBS)

In order to determine whether a quercetin liposomal formulation would be stable in an in vivo environment, the stability of quercetin-loaded liposomes were examined in the presence of fetal bovine serum (serum). Quercetin concentration was measured by a UV-Vis spectrophotometer based on absorbance at 372 nm. The liposome formulation (400 4) after concentration was added to 1.6 mL of fetal bovine serum (FBS, Gibco, Burlington, ON, Canada) and the FBS/liposomal quercetin mixture was placed in a 37° C. water bath for 24 hours.

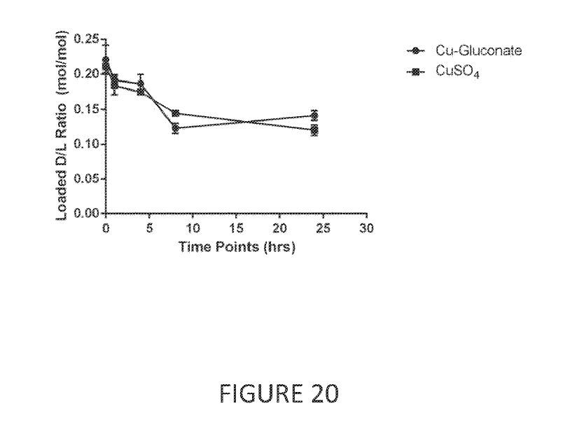

When incubated in fetal bovine serum (FBS) at 37° C., the drug-to-lipid ratio of copper sulfate liposomes dropped 17% after one hour, 25% after eight hours, and 44% after 24 hours incubation (FIG. 20). Quercetin-loaded copper gluconate liposomes showed a similar FBS release profile (FIG. 20).

### Example 16

### Pharmacokinetics Profile of Quercetin Encapsulated into CuSO₄ and Copper Gluconate Liposomes

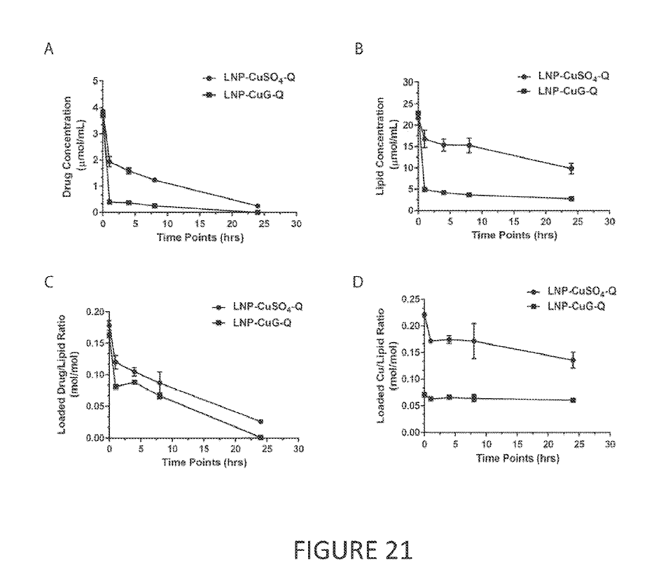

RAG2m mice were injected with a single dose of either quercetin liposomal formulation (CuSO₄ or CuG) at 50 mg/kg. With the quercetin/CuSO₄ liposome formulation, plasma concentrations of quercetin decreased by approximately 50% (3.87 μmol/mL) at 1 hour post-injection (FIG. 21A). At 24 hours post-injection, about 6.4% of the injected quercetin remained in the plasma compartment (FIG. 21A). In contrast, lipid concentrations displayed less of a decrease than quercetin concentrations with a decrease of 28% at 1 hour post-injection (15.71 μmol/mL) and 55% at 24 hours post-injection (9.78 μmol/mL) (FIG. 21B). Examination of the drug-to-lipid ratio showed a decreasing trend from 0.12 (mol/mol) at the 1 hour time-point to 0.03 (mol/mol) at the 24 hour time-point (FIG. 21C). However, copper-to-lipid ratios did not change significantly across 24 hours (FIG. 21D). These results demonstrate the release of quercetin, not copper-quercetin, from liposomes in vivo.

For quercetin liposomes with internal copper gluconate (CuG), the plasma concentration of quercetin decreased by 92.8% within 1 hour following injection (FIG. 21A). Lipid concentrations decreased by 78.72% at 1 hour post-injection and by 87.79 at 24 hours post-injection (FIG. 21B). The drug-to-lipid ratio showed a trend going from 0.08 (mol/mol) at 1 hour post-injection to 0.001 (mol/mol) at 24 hours post-injection (FIG. 21C). However, copper-to-lipid ratios remained consistent (approximately 0.07 mol/mol) over a 24-hour period indicating that quercetin was released from liposomes as a free agent (FIG. 21D).

### Example 17

### Cytotoxic Effects of Clioquinol (Copper Dependant and Independent)

Clioquinol is an analogue of 8-hydroxyquinoline and has been used as an anti-fungal agent in the clinic. It is also an anti-cancer agent when complexed with copper. It has been reported that a copper clioquinol (Cu(CQ)₂) complex behaves as a proteosome inhibitor and metal ionophore.

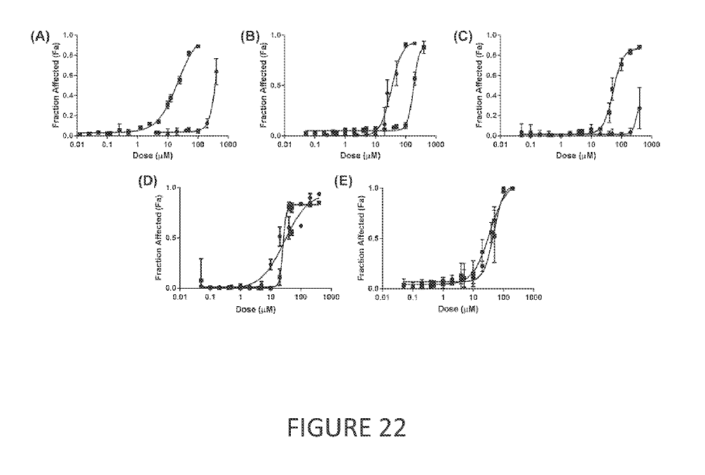

The anticancer activity of clioquinol (CQ) in cancer lines through copper dependent and independent pathways was examined. Both CQ (-●-) and Cu(CQ)₂ (-▪-) were dissolved in DMSO and diluted to a final concentration of <0.5% (at higher concentrations (>100 μM) of Cu(CQ)₂, precipitated drug could be seen under the microscope). A2780-S (human ovarian carcinoma, platinum sensitive), A2780-CP (human ovarian carcinoma, platinum insensitive) as well as A549 (human lung cancer), U251 (human glioblastoma) cytotoxicity curves (FIG. 22 A-D) were obtained with the IN CELL Analyzer 2200. Cell viability was assessed based on loss of plasma membrane integrity 72 hours following treatment. Total cell count and dead cell count were determined using Hoechst 33342 and ethidium homodimer staining, respectively. The CQ cytotoxicity curve in MV-4-11 (human leukemia) was generated using PrestoBlue reagent to establish cell viability through metabolic activity.

As shown in FIG. 22, clioquinol is cytotoxic to cancer cells and the activity can be copper dependant (A-C) or copper independent (D/E). In particular, CQ activity was found to be copper dependant in A2780-S, A2780-CP and A549 but copper independent in U251 and MV-4-11 cells.

### Example 18

### Encapsulation and In Vitro Retention of Copper Clioquinol in Liposomes

This example shows that clioquinol (CQ) can be encapsulated and retained in copper containing liposomes through metal complexation.

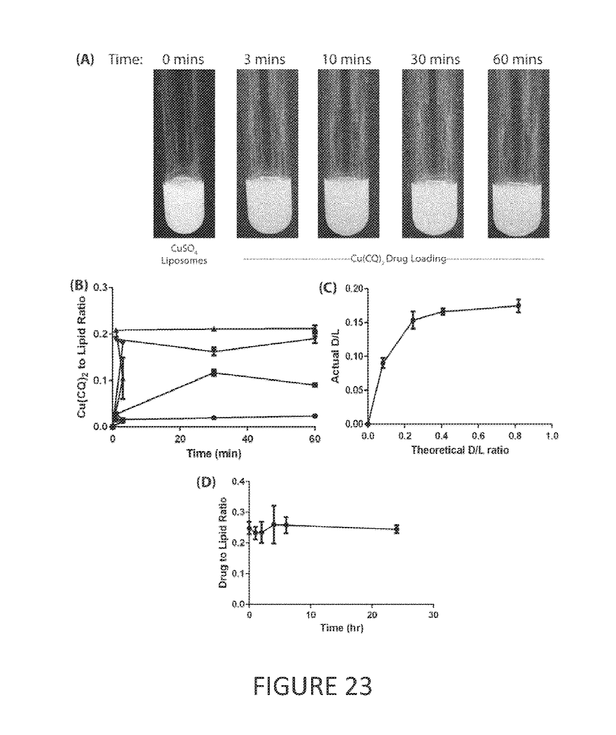

The complexation reaction can be visualized by a colour change (white to yellow) as time elapses. As shown in FIG. 23A, the contents of test-tubes containing CQ and copper at different time points are darker in colour moving from left to right (0, 3, 10, 30 and 60 mins).

Under the conditions examined, the maximum encapsulation of CQ in the liposomes was found at a temperature of at least 40° C. (FIG. 23B). The encapsulation was performed through the addition of CQ as a solid powder owing to its poor water solubility and unencapsulated drug was removed using a Sephadex G50 column. Although the liposome loading was carried out by the addition of the CQ in the form of a powder, CQ can be dissolved in a solvent and added to the external solution of copper-containing liposomes as well. The CQ would then pass through the bilayer and into the internal solution of the liposome where complexation occurs.

The maximum CQ that can be complexed is correlated to the amount of copper that is entrapped as seen in FIG. 23C. The Cu(CQ)₂ formulation did not show significant release of its contents at 37° C. in 80% fetal bovine serum (FBS) over 24 hrs.

### Example 19

### Pharmacokinetics of Copper Clioquinol Encapsulated in Liposomes

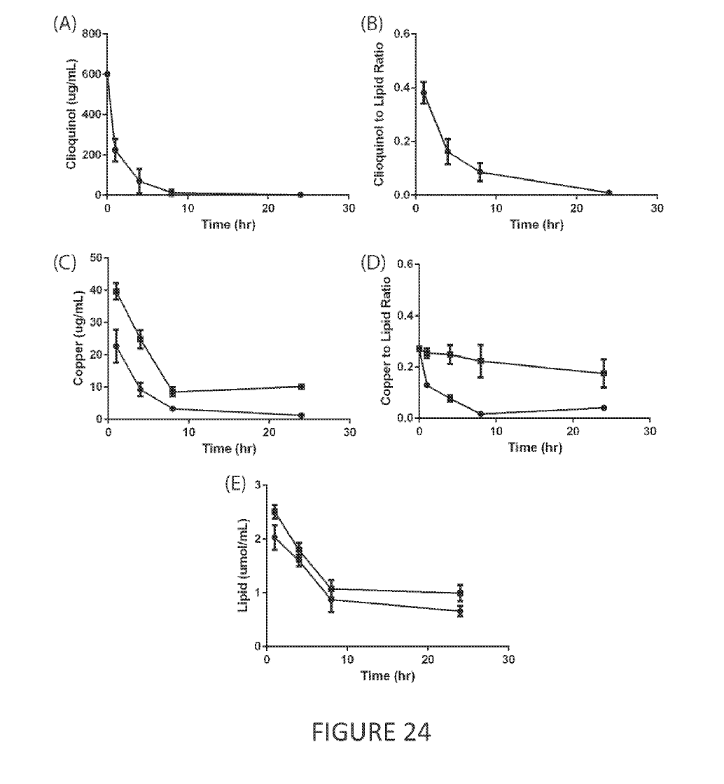

The Cu(CQ)₂ complex elimination profile was characterized and compared to 300 mM copper sulfate-containing liposomes. Clioquinol elimination can be seen in FIG. 24A, wherein at 24 hours the amount of CQ in the plasma compartment is undetectable. The CQ to lipid ratio is shown in FIG. 24B and indicates that the CQ is releasing from the liposome and by 24 hrs no CQ is associated with the liposome. Copper elimination and copper-to-lipid ratio are given in FIG. 24C and D and it can be seen that both formulations show similar copper elimination. The copper-to-lipid ratio of the Cu(CQ)₂ liposome approaches zero, while the copper-to-lipid ratio of copper-containing liposomes remains above 0.2. This is indicative that CQ leaves the liposome as a copper complex and that, in the absence of the Cu(CQ)₂ complex, copper remains associated with the liposome. The lipid elimination of both liposomal preparations is identical, suggesting that differences in the Cu-to-lipid ratio are a result of copper release from the liposome and not a result of differences in lipid elimination.

### Example 20

### The In Vivo Activity of Copper Clioquinol Encapsulated in Liposomes

Through complexing CQ inside the liposome with copper, a formulation was created that is injectable. Cu(CQ)₂ was injected at 15 mg/kg intraperitoneally (i.p.) once daily Monday to Friday for 2 weeks or intravenously (i.v.) at 30 mg/kg Monday, Wednesday, and Friday for 2 weeks.

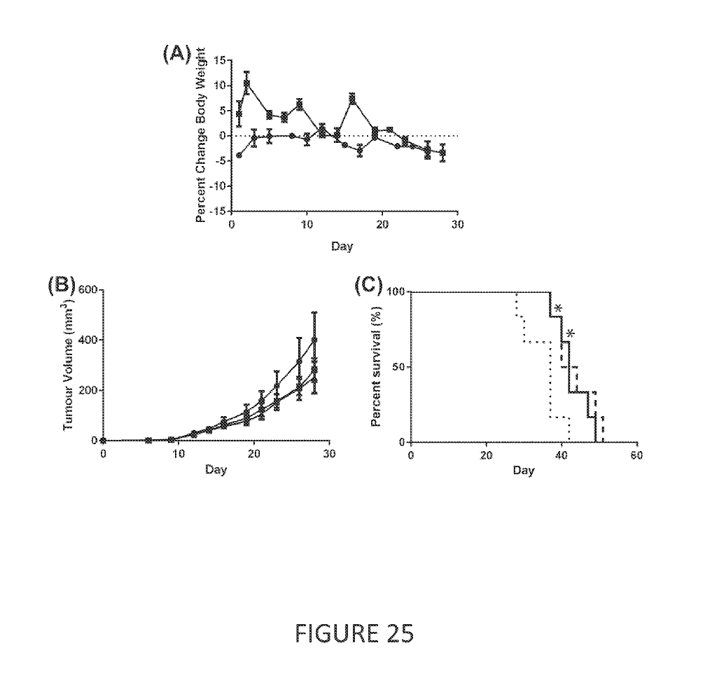

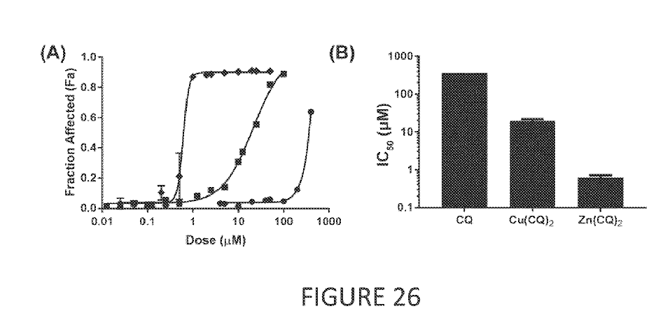

Cu(CQ)₂ administered i.p and i.v. were both tolerated well with no weight loss >5%. The Cu(CQ)₂ was tested in a U251 subcutaneous tumour model. Mice were implanted with 1×10⁶ cells then treated when tumours were 50-100 mm³. There was no significant difference in tumour growth between the vehicle and the Cu(CQ)₂ treated groups (FIG. 25B) but a statistically significant increase in survival was seen for both treatment groups (FIG. 25C).

The method described here allows for the preclinical development of Cu(CQ)₂. Cu(CQ)₂ is tolerated at doses that can result in significant increases in survival.

### Example 21

### Zn(CQ)₂ Complex Toxicity

Clioquinol is able to form complexes with divalent metal ions besides copper. Copper enhances the activity of CQ when administered to cancer cells as a complex. This complex is insoluble and was dissolved in DMSO to a final concentration of 0.5%. Similarly, the zinc complex of CQ is insoluble and is more active than CQ and Cu(CQ)₂.

These results show that other metal complexes can be prepared with clioquinol that exhibit cytotoxicity.

### Example 22

### The Encapsulation of Poorly Soluble Therapeutic Agents in Copper-Containing Liposomes can Enhance their In Vivo Activity

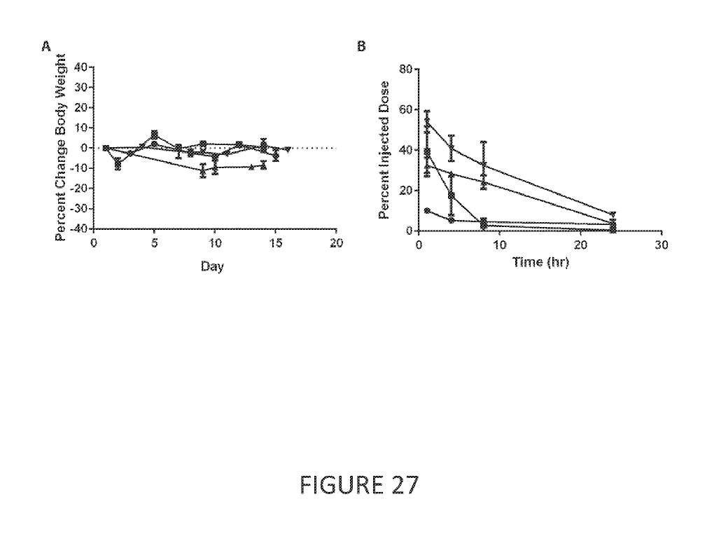

This example summarizes the in vivo activity of the therapeutic agents in the foregoing examples complexed with copper and encapsulated in liposomes. The formulations examined include liposomal Cu(DDC)₂, Cu(CQ)₂, CuQu and Cu-CX5461. FIG. 27 shows the results.

Liposomal formulations described in Example 5 (Cu(DDC)₂, Cu(CQ)₂, CuQu and Cu-CX5461) were prepared for single dose safety studies in mice and once a safe dose was defined, the elimination of the copper complex compound was determined as described in the methods above.

FIG. 27A summarizes the change in body weight of mice injected with the indicated formulation at the determined maximum tolerated dose. The formulations caused <15% body weight loss and other health status indicators suggested only mild and reversible changes in animal health status.

The elimination behaviors of the intravenously injected compounds are shown in FIG. 27B. Cu(CX5461) exhibited the longest circulation longevity with almost >30% of the injected dose remaining in circulation after 8 hr.

### Example 23

### Cytotoxicity Studies of Drug Combinations

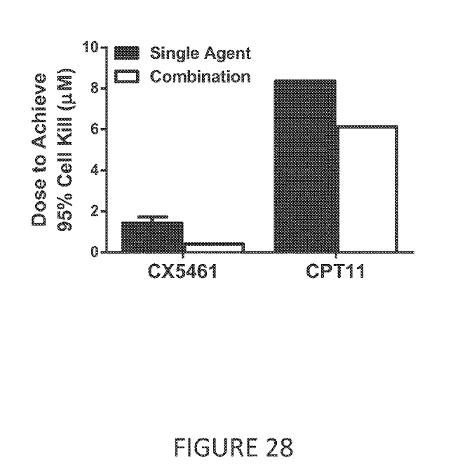

Cytoxocity studies were conducted using CX5461 and CPT11 alone and in combination. The results are presented in FIG. 28.

The dose response curves for CX5461 and irinotecan (CPT11) as single agents against MV-4-11 (leukemia) cells were first generated. A consistent molar ratio of 1:15 (CX5461: CPT11) was found at IC₁₀, IC₅₀, and IC₉₀. This fixed ratio was then used to generate a dose response curve for the CX5461 and CPT11 combination. The resulting data were processed through the CompuSyn software which utilizes the Chou-Talalay method to calculate combination indices (CI), where CI<1 indicates synergistic effects. With this particular combination, the CI was 0.82 at a fraction affected of 95%. As shown, this suggests that both drugs can be used at the same time at much lower doses to achieve 95% cell death, which is favourable in terms of improved therapeutic activity and reduced toxicity.

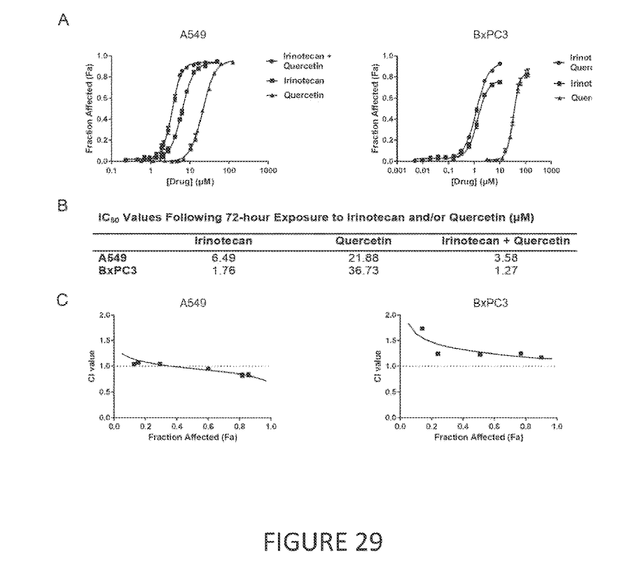

Cytotoxicity curves were also generated for quercetin and irinotecan. The results are shown in FIG. 29.

The cytotoxic effects of quercetin and/or irinotecan (CPT11) were investigated in A549 and BxPC3 cells (FIG. 29A). Quercetin and CPT11 were added at ratios of 1:2.5 (CPT11:Quer) for A549 and 1:18 (CPT11:Quer) for BXPC3. The fixed drug ratios were empirically determined by calculating the ratios of the single agents at equi-toxic doses in each cell line. The ratio that was maintained across the middle portion of the sigmoidal dose response curve was used in the combination studies. The dose response curve for the combination studies was plotted against concentrations of the more potent agent, CPT11. After 72 hours of exposure, the IC₅₀ for the combination treatments were 3.58 μM for A549 and 1.27 μM for BxPC3 (FIG. 29B). As indicated by combination indices (CI), quercetin and CPT11 displayed synergy at high effect levels (>60% cell kill) for A549 but the two agents acted antagonistically at all effect levels for BxPC3 (FIG. 29C).

The invention has been described with reference to one or more examples and embodiments described above. However, the examples and embodiments are exemplary only and the invention is defined solely by the claims appended herein.

1-43. (canceled)

44. A pharmaceutical formulation for delivery of a poorly soluble therapeutic agent, the formulation comprising:
a pre-formed liposome comprising a metal ion and the poorly soluble therapeutic agent, wherein the poorly soluble therapeutic agent has a solubility of less than 1 mg/mL either in water or in a solution of the metal ion,
the therapeutic agent comprises a metal complexation moiety, and
the complexation moiety complexes with the metal ion.

45. The pharmaceutical formulation of claim 44, wherein the poorly soluble therapeutic agent has a basic pKₐ of at least 8.

46. The pharmaceutical formulation of claim 44, wherein the poorly soluble therapeutic agent is non-pH gradient loadable into the pre-formed liposome.

47. The pharmaceutical formulation of claim 44, wherein the poorly soluble therapeutic agent has a solubility when in water that is less than 1 mg/mL or a solubility of less than 1 mg/mL when in a solution of the metal ion having a concentration between 100 mM to 500 mM.

48. The formulation of claim 44, wherein the poorly soluble therapeutic agent comprising the metal complexation moiety is selected from the group consisting of clioquinol, diethyldithiocarbamate, quercetin and CX5461.

49. The formulation of claim 44, wherein the poorly soluble therapeutic agent is CX3543.

50. The formulation of claim 44, wherein the poorly soluble therapeutic agent is one of two or more different therapeutic agents that are present in the formulation, and wherein the two or more therapeutic agents are each encapsulated in the same or different liposomes in the formulation.

51. The formulation of claim 50, wherein one of the two therapeutic agents has a solubility in at least one of water and a metal-containing solution that is at least 1 mg/mL.

52. The formulation of claim 44, wherein the metal ion is a transition metal.

53. The formulation of claim 44, wherein the metal ion is a Group IIIb metal.

54. The formulation of claim 52, wherein the transition metal is copper or zinc.

55. A method for producing a pharmaceutical formulation for delivery of a poorly soluble therapeutic agent, the method comprising:
(i) providing a pre-formed liposome comprising a phospholipid bilayer and a metal ion that complexes with the poorly soluble therapeutic agent;
(ii) providing a poorly soluble therapeutic agent in the solution external to the pre-formed liposome, the therapeutic agent comprising a metal ion complexation moiety; and
(iii) allowing the poorly soluble therapeutic agent to move across the phospholipid bilayer of the pre-formed liposome into an internal solution of the liposome,
wherein the poorly soluble therapeutic agent has a solubility of less than 1 mg/mL either in water or a solution containing the metal ion.

56. The method of claim 55, wherein the metal ion is a Group IIIb metal.

57. The method of claim 55, wherein the metal is copper or zinc.

58. The method of claim 55, wherein the poorly soluble therapeutic agent has a pKₐ of at least 8.

59. The method of claim 55, wherein the poorly soluble therapeutic agent is non-pH gradient loadable into the pre-formed liposome.

60. The method of claim 55, wherein the poorly soluble therapeutic agent has a solubility when in water that is less than 1 mg/mL or wherein the poorly soluble therapeutic agent has a solubility of less than 1 mg/mL when in a solution of the metal ion having a concentration between 100 mM and 500 mM.

61. The method of claim 55, wherein a solution external to the pre-formed liposome contains substantially no metal ions that complex with the poorly soluble therapeutic agent or comprises a chelating agent that chelates with the metal ions.

62. A pharmaceutical formulation produced by the method of claim 55.

63. A pre-formed liposome comprising a therapeutic agent selected from the group consisting of clioquinol, diethyldithiocarbamate, quercetin and CX5461, wherein the pre-formed liposome comprises a metal ion that complexes with the therapeutic agent.
